(19) United States
(12) Patent Application Publication
(10) Pub. No.: US 2025/0205322 A1
(43) Pub. Date: Jun. 26, 2025

(54) MHC IB-MEDIATED AQUAPORIN 4 (AQP4)-SPECIFIC IMMUNOSUPPRESSION AS A NOVEL TREATMENT FOR NMO

(71) Applicant: Julius-Maximilians-Universität Würzburg, Würzburg (DE)

(72) Inventors: Valentin BRUTTEL, Würzburg (DE); Jörg WISCHHUSEN, Würzburg (DE); Shriya Mamatha JAYARAM, Würzburg (DE); Fadhil AHSAN, Berlin (DE)

(73) Assignee: Julius-Maximilians-Universität Würzburg, Würzburg (DE)

(21) Appl. No.: 18/849,075

(22) PCT Filed: Mar. 24, 2023
(86) PCT No.: PCT/EP2023/057684

§ 371 Date: Sep. 20, 2024

(30) Foreign Application Priority Data
Mar. 24, 2022 (EP) 22164161.6

(51) Int. Cl. A61K 39/00; A61P 25/00; A61P 37/06; C07K 14/705; C07K 14/74

(52) U.S. Cl. CPC A61K 39/0008; A61P 25/00; A61P 37/06; C07K 14/705; C07K 14/70539; A61K 2039/577; A61K 2039/605; C07K 2319/40; C07K 2319/50

(57) ABSTRACT

The present invention relates to therapeutical uses of non-classical human major histocompatibility complex (MHC) molecules (also named MHC class Ib molecules) in combination with peptide antigens for the treatment of neuromyelitis optica (NMO). The invention more specifically relates to recombinant polypeptides comprising peptide antigens and one or more domains of a non-classical MHC class Ib molecule. The invention also relates to methods of producing such recombinant polypeptides, pharmaceutical compositions comprising the same, as well as their uses for treating neuromyelitis optica (NMO).

### FIELD OF THE INVENTION

The present invention relates to therapeutical uses of non-classical human major histocompatibility complex (MHC) molecules (also named MHC class Ib molecules) in combination with peptide antigens for the treatment of neuromyelitis optica (NMO). The invention more specifically relates to recombinant polypeptides comprising peptide antigens and one or more domains of a non-classical MHC class Ib molecule. The invention also relates to methods of producing such recombinant polypeptides, pharmaceutical compositions comprising the same, as well as their uses for treating neuromyelitis optica (NMO).

### BACKGROUND

Neuromyelitis optica (NMO), a demyelinating autoimmune disease of the central nervous system (CNS) with an incidence between 0.05 and 0.4/100,000. It is characterized by similar symptoms as Multiple Sclerosis. However, whereas Multiple Sclerosis is often characterized by a relapsing-remitting couse of disease, patients with NMO rarely experience remissions. Moreover, some treatments that are beneficial in patients with MS fail in NMO or may even aggravate the disease. Therefore, NMO is more difficult to treat than MS and usually progresses faster Current therapeutic strategies for NMO focus on relapse prevention or treatment of acute disease relapses. Acute relapses are usually treated with immunosuppressive therapeutics such as glucocorticoids or plasmapheresis to remove autoreactive antibodies. More recently, therapeutic antibodies that inhibit the complement system (eculizumab) or block inflammatory cytokines (satralizumab) have also been approved.

However, not only autoreactive but also protective immune functions are inhibited. These are essential to protect the patient from viruses, bacteria or tumors. Accordingly, often only partial inhibition can be targeted. Since autoimmune diseases usually worsen after recurrent attacks, a great deal of effort is put into relapse prevention. However, a general slow progression of this autoimmune disease can only be slowed but not prevented. To circumvent this dilemma, attempts have been made for some time to induce immunological tolerance to antigens attacked by autoreactive immune cells (antigen-specific immunosuppression, ASI).

Two strategies have been evaluated so far, at least in early clinical studies. Similar to hyposensitization strategies in allergy, large amounts of antigens have been administered by different routes to induce antigen-specific tolerance. However, in autoimmune diseases, these strategies have caused severe side effects and have not been clinically successful. Attempts to induce tolerance by adoptive transfer of antigen-specific regulatory T cells or antigen-loaded tolerogenic dendritic cells appear more promising. However, these strategies are extremely complex and expensive, requiring GMP-compliant production and quality control processes for each individual patient. Therefore, even if small clinical trials have been successful, it is highly questionable whether adoptive transfer therapies will be available for many patients in the foreseeable future. WO 2018/215340 relates to combinations of MHC class Ib molecules and peptides for targeted therapeutic immunomodulation.

Taken together, there remains a need for improved drugs for the treatment of neuromyelitis optica (NMO).

### DESCRIPTION OF THE INVENTION

The inventors have found that human MHC class Ib molecules such as HLA-G possess the ability to induce antigen-specific tolerance towards presented peptide antigens. Thus, albeit being of similar structure and sequence as classical human MHC class Ia molecules which induce antigen peptide-specific immune responses, MHC class Ib molecules can advantageously be used according to the invention to suppress immune responses in an antigen-specific manner. Additionally, the inventors have found that for the suppression of immune responses according to the invention, molecules other than naturally occurring MHC class Ib molecules, and in particular polypeptides which only comprise at least one domain of an MHC class Ib molecule, preferably at least an [alpha]3 domain of an MHC class Ib molecule, can be used: The [alpha]1 and [alpha]2 domains of variable class I a molecules can be combined with the [alpha]3 domain of a human MHC class Ib molecule in order to suppress immune responses towards peptides presented by these antigens.

Antigen-loaded HLA-G molecules can be unstable. Thus, the inventors designed soluble recombinant polypeptides comprising a peptide antigen, an MHC class Ib molecule such as HLA-G and 32-microglobulin (b2m), and connected these three components covalently (e.g., via covalent linkers). Alternatively, the antigen-binding α1 and α2 domains of an MHC class Ib molecule such as HLA-G were exchanged by the respective domains of other MHC molecules to enhance the flexibility and versatility of these recombinant polypeptides (see, for instance, FIG. 2). These alternative recombinant polypeptides can be designed with antigen-binding domains of other human HLA molecules. It was previously found that constructs comprising the α1 and α2 domains of murine H2-K^{b} can present the ovalbumin-derived peptide SIINFEKL to OT-1 T cells. (OT-1 T cells express a transgenic T cell receptor that specifically recognizes this antigen) (WO 2018/215340).

Surprisingly, the inventors have found that by using the recombinant polypeptides of the invention, immune responses against neuroinflammatory autoantigens can be suppressed and cells that induce tolerance to human aquaporin 4 (AQP4) can be induced. Further, using established models for neuroinflammatory conditions, the inventors have shown that surrogates of these polypeptides (adapted for use in mice) can be used to treat neuroinflammatory diseases. Thus, according to the invention, neuromyelitis optica (NMO) can be treated by the recombinant polypeptides of the invention.

Experimental data of the inventors show that a suitable peptide antigen and the presence of an [alpha]3 domain of an MHC class Ib molecule (e.g. HLA-G) are required to achieve the desired effect. Therefore, this approach goes beyond previously described strategies that either use antigenic peptides in the absence of costimulation (resulting in anergic rather than tolerogenic T cells), or MHC class Ib molecules in an antigen-unspecific setting.

Moreover, according to the invention, the recombinant polypeptides of the invention do not only modulate T-cell responses but also prevent the formation of aquaporin 4 (AQP4)-specific autoantibodies. It is expected that this advantage will translate into a clinical improvement in human patients having neuromyelitis optica (NMO), because aquaporin 4 (AQP4)-specific autoantibodies are involved in the pathology of neuromyelitis optica (NMO).

Accordingly, the invention relates to the following preferred embodiments:
A recombinant polypeptide capable of presenting a peptide antigen, the recombinant polypeptide comprising, in an N- to C-terminal order,
i) a peptide antigen presented by said recombinant polypeptide, wherein the peptide antigen is a peptide of human aquaporin 4;
ii) optionally a linker sequence;
iii) optionally a sequence of a human polypeptide domain comprising a sequence of a human β2 microglobulin, or an amino acid sequence at least 90% identical to the amino acid sequence of human β2 microglobulin represented by SEQ ID NO: 5;
iv) optionally a linker sequence;
v) optionally an [alpha] 1 domain of an MHC molecule;
vi) optionally an [alpha] 2 domain of an MHC molecule;
vii) an [alpha] 3 domain of an MHC class Ib molecule or a derivative of an [alpha] 3 domain of an MHC class Ib molecule, said derivative being capable of binding to ILT2 or ILT4;
viii) optionally a protease cleavage site;
ix) optionally a spacer sequence; and
x) optionally an affinity tag.
1. The recombinant polypeptide according to item 1, wherein said peptide antigen according to i) is 7 to 11 amino acids in length, preferably 8-10 amino acids in length.
2. The recombinant polypeptide according to item 1 or 2, wherein said peptide antigen according to i) consists of an amino acid sequence selected from the group consisting of the amino acid sequences of SEQ ID NOs: 2, 22, 23 and 24.
3. The recombinant polypeptide according to any one of the preceding items, wherein said peptide antigen consists of the amino acid sequence of SEQ ID NO: 2 or SEQ ID NO: 22.
4. The recombinant polypeptide according to any one of items 1-4, wherein said peptide antigen consists of the amino acid sequence of SEQ ID NO: 2.
5. The recombinant polypeptide according to any one of items 1-3, wherein said peptide antigen consists of the amino acid sequence of SEQ ID NO: 23 or SEQ ID NO: 24.
6. The recombinant polypeptide according to any one of the preceding items, wherein said [alpha]1 domain according to (v) and said [alpha]2 domain according to (vi) are from a human MHC class Ia molecule or from a human MHC class Ib molecule.
7. The recombinant polypeptide according to any one of the preceding items, wherein said [alpha]1 domain according to (v) and said [alpha]2 domain according to (vi) are from a human MHC class Ia molecule.
8. The recombinant polypeptide according to item 8, wherein said [alpha]1 domain according to (v) and said [alpha]2 domain according to (vi) are from a human HLA-A2 molecule.
9. The recombinant polypeptide according to item 7, wherein said [alpha]1 domain according to (v) and said [alpha]2 domain according to (vi) are from a human MHC class Ib molecule.
10. The recombinant polypeptide according to item 10, wherein said [alpha]1 domain according to (v) and said [alpha]2 domain according to (vi) are from a human HLA-G molecule.
11. The recombinant polypeptide according to any one of the preceding items, wherein the [alpha] 3 domain of the MHC class Ib molecule according to (vii) is an [alpha] 3 domain of human HLA-E, human HLA-F or human HLA-G.
12. The recombinant polypeptide according to any one of the preceding items, wherein the [alpha] 3 domain of the MHC class Ib molecule according to (vii) is an [alpha] 3 domain of human HLA-G.
13. The recombinant polypeptide according to any one of the preceding items, wherein the [alpha]3 domain or derivative according to (vii) is identical to or has at least 80% amino acid sequence identity, preferably at least 90% amino acid sequence identity, with the [alpha]3 domain having the amino acid sequence of SEQ ID NO: 9 or SEQ ID NO: 21.
14. The recombinant polypeptide according to item 14, wherein the [alpha]3 domain or derivative according to (vii) is identical to or has at least 92% amino acid sequence identity with the [alpha]3 domain having the amino acid sequence of SEQ ID NO: 9 or SEQ ID NO: 21.
15. The recombinant polypeptide according to item 14, wherein the [alpha]3 domain or derivative according to (vii) is identical to or has at least 94% amino acid sequence identity with the [alpha]3 domain having the amino acid sequence of SEQ ID NO: 9 or SEQ ID NO: 21.
16. The recombinant polypeptide according to item 14, wherein the [alpha]3 domain or derivative according to (vii) is identical to or has at least 96% amino acid sequence identity with the [alpha]3 domain having the amino acid sequence of SEQ ID NO: 9 or SEQ ID NO: 21.
17. The recombinant polypeptide according to item 14, wherein the [alpha]3 domain or derivative according to (vii) is identical to or has at least 98% amino acid sequence identity with the [alpha]3 domain having the amino acid sequence of SEQ ID NO: 9 or SEQ ID NO: 21.
18. The recombinant polypeptide according to item 14, wherein the [alpha]3 domain or derivative according to (vii) is identical to or has at least 99% amino acid sequence identity with the [alpha]3 domain having the amino acid sequence of SEQ ID NO: 9 or SEQ ID NO: 21.
19. The recombinant polypeptide according to item 14, wherein the [alpha]3 domain according to (vii) is identical to the [alpha]3 domain having the amino acid sequence of SEQ ID NO: 9 or SEQ ID NO: 21.
20. The recombinant polypeptide according to any one of the preceding items, wherein the linker sequence according to (ii) and/or the linker sequence according to (iv) comprises the amino acid sequence (GGGGS)n, wherein n is an integer equal to or higher than 1.
21. The recombinant polypeptide according to item 21, wherein the linker sequence according to (ii) comprises the amino acid sequence (GGGGS)n, and wherein n is an integer selected from the group consisting of 1, 2, 3, 4, 5, 6, 7, 8, 9 and 10 and is preferably selected from the group consisting of 2, 3, 4 and 5.
22. The recombinant polypeptide according to item 21 or 22, wherein the linker sequence according to (iv) comprises the amino acid sequence (GGGGS)n, and wherein n is an integer selected from the group consisting of 1, 2, 3, 4, 5, 6, 7, 8, 9 and 10 and is preferably selected from the group consisting of 2, 3, 4 and 5.
23. The recombinant polypeptide according to any one of the preceding items, wherein said sequence of a human polypeptide domain according to (iii) is at least 95% identical to the amino acid sequence of SEQ ID NO: 5, preferably at least 98% identical to the amino acid sequence of SEQ ID NO: 5 and more preferably identical to the amino acid sequence of SEQ ID NO: 5.
24. The recombinant polypeptide according to any one of the preceding items, wherein said polypeptide is dimeric or multimeric.
25. The recombinant polypeptide according to any one of the preceding items, wherein the polypeptide comprises or consists of all of the components i) to vii)
26. The recombinant polypeptide according to any one of the preceding items, wherein the polypeptide does not comprise components viii) to x).
27. The recombinant polypeptide according to any one of items 1 to 26, wherein the polypeptide comprises or consists of all of the components i) to x).
28. The recombinant polypeptide according to any one of the preceding items, further comprising an N-terminal secretion signal peptide sequence.
29. The recombinant polypeptide according to any one of items 1-28, wherein the recombinant polypeptide consists of an amino acid sequence consisting of the following ((a) and (b)) in an N to C-terminal order:
   (a) a peptide antigen selected from the group consisting of the amino acid sequences of SEQ ID NOs: 2, 22, 23 and 24, and
   (b) the amino acid sequence of SEQ ID NO: 16.
30. The recombinant polypeptide according to any one of the preceding items, wherein the recombinant polypeptide is soluble.
31. A nucleic acid encoding one or more polypeptides according to any one of the preceding items.
32. The nucleic acid according to item 32, wherein the nucleic acid is a vector.
33. A pharmaceutical composition or kit comprising at least one nucleic acid according to items 32 or 33.
34. A pharmaceutical composition or kit comprising at least one recombinant polypeptide according to any one of items 1-31.
35. The pharmaceutical composition or kit according to item 35, wherein the pharmaceutical composition or kit comprises at least two different recombinant polypeptides according to any one of items 1-31, and wherein each of the different polypeptides comprises a different peptide antigen as defined in any one of items 3 to 6.
36. A pharmaceutical composition or kit according to any one of items 34-36, for use in the treatment of neuromyelitis optica in a human patient.
37. The pharmaceutical composition or kit for use according to item 37, wherein the treatment is treatment by immunotherapy.
38. The pharmaceutical composition or kit for use according to any one of items 37-38, wherein the treatment is by inducing immunological tolerance against human aquaporin 4.
39. The pharmaceutical composition or kit for use according to any one of items 37-39, wherein the treatment is for reducing plasma or cerebrospinal fluid levels of autoantibodies against human aquaporin 4.
40. The pharmaceutical composition or kit for use according to any one of items 37-40, wherein the human patient is a patient who had plasma or cerebrospinal fluid autoantibodies against human aquaporin 4 prior to the start of the treatment.
41. The pharmaceutical composition or kit for use according to any one of items 37-41, wherein the treatment is by inducing myelin-specific regulatory T cells.
42. A recombinant host cell comprising a nucleic acid or a vector according to item 32 or 33 and expressing the recombinant polypeptide according to any one of items 1-31.
43. A method for obtaining pharmaceutical composition comprising a polypeptide according to any one of items 1-31, the method comprising the steps of (a) culturing the recombinant host cell of item 43 under conditions allowing expression of the recombinant polypeptide from the nucleic acid molecule, (b) recovering the recombinant polypeptide, (c) purifying the recombinant polypeptide, and (d) formulating the recombinant polypeptide into a pharmaceutical composition.

### BRIEF DESCRIPTION OF THE DRAWINGS

FIG. 1: Depiction of a peptide-loaded soluble MHC Ib molecule suitable to achieve therapeutic antigen-specific immunomodulation.

The presented peptide antigen is depicted in dotted spheres, the HLA-G alpha1-3 domains are sketched in light-grey, and the beta2microglobulin domain is shown in dark grey. An optional linker connecting the antigenic peptide with the beta2microglobulin molecule is displayed in grey stick style, and an optional disulfide trap is depicted in black spheres. This figure was generated using Pymol and is adapted from structures published in Clements et al., Proc Natl Acad Sci USA. 2005 Mar. 1; 102(9):3360-5 and Hansen et al., Trends Immunol. 2010 October; 31(10):363-9.

FIG. 2: Example for a vector-based construct encoding a single chain MHC Ib molecule suitable for therapeutic peptide-specific immunomodulation.

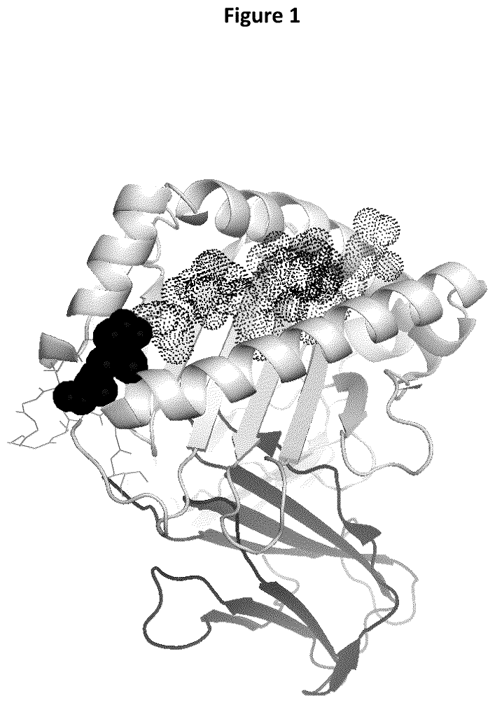

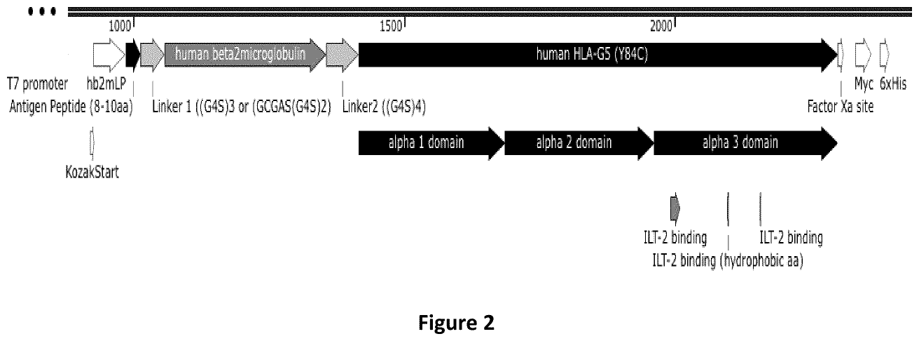

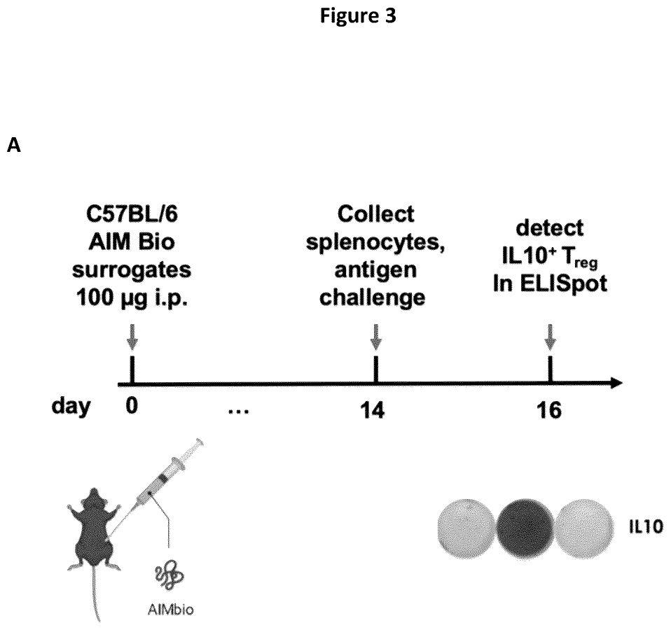

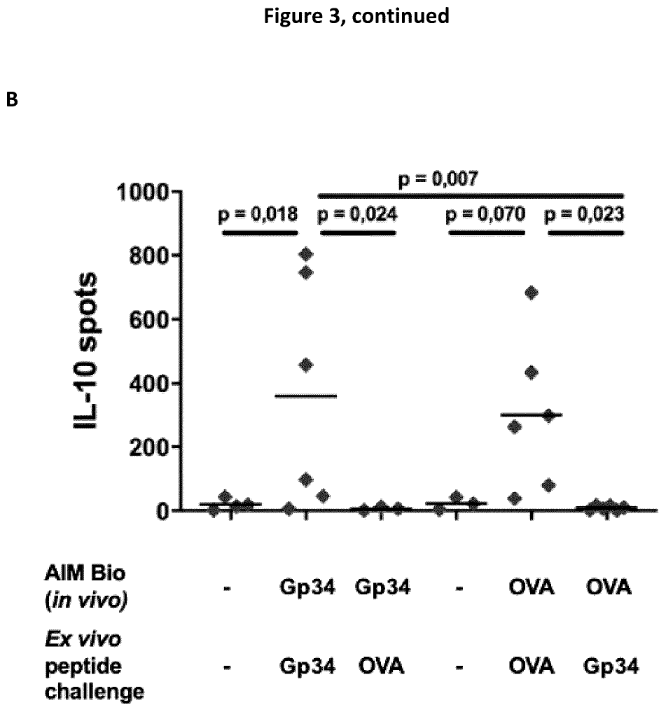

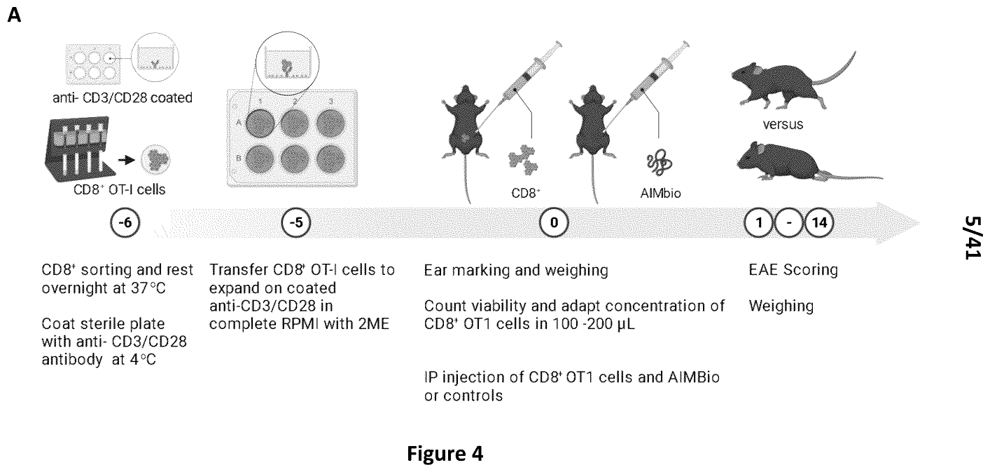

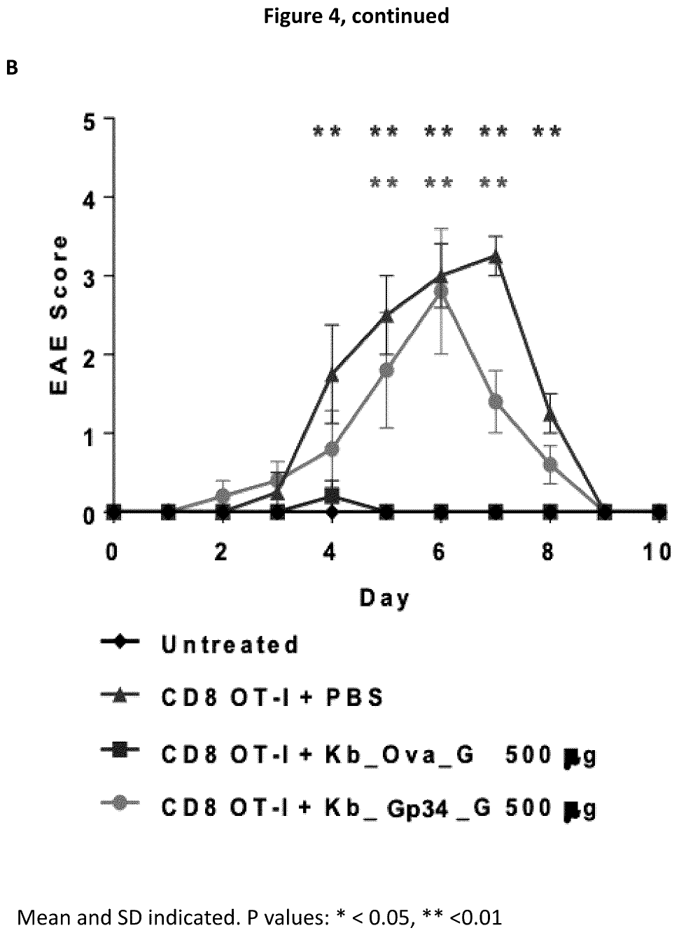

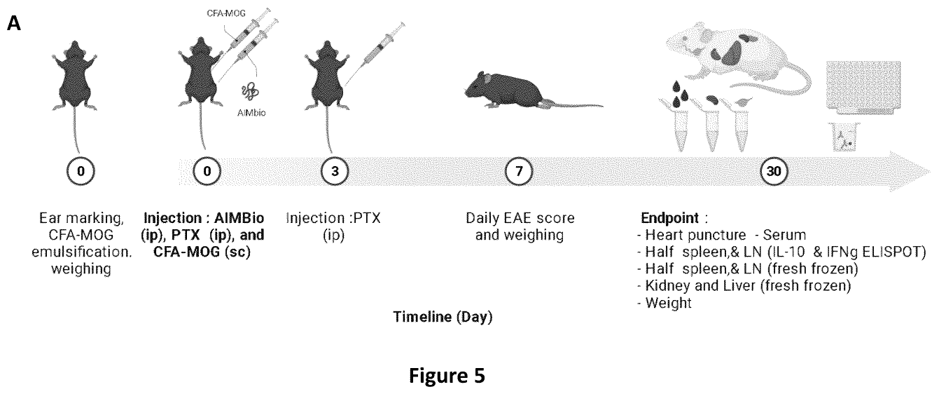

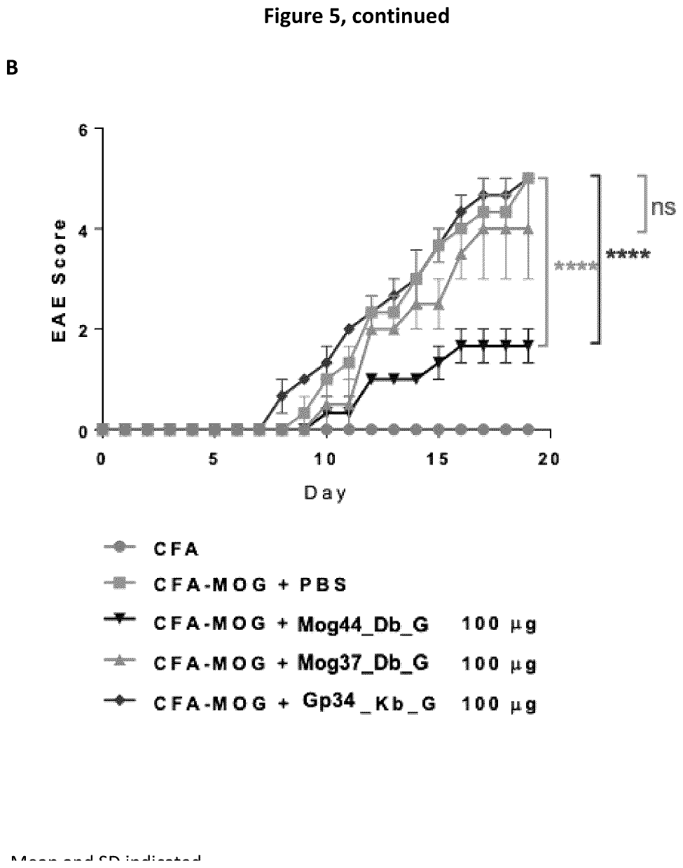

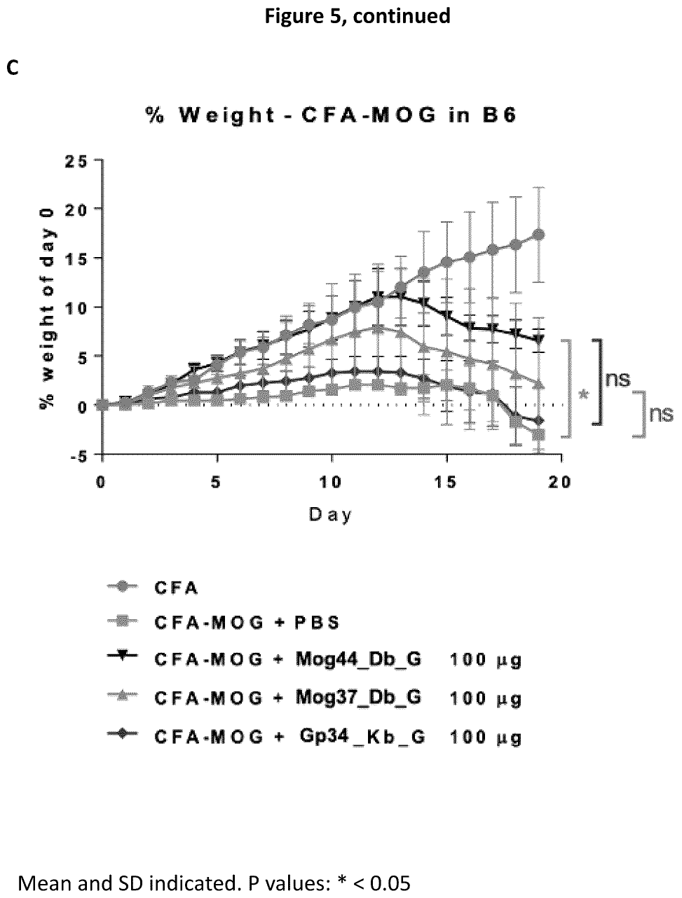

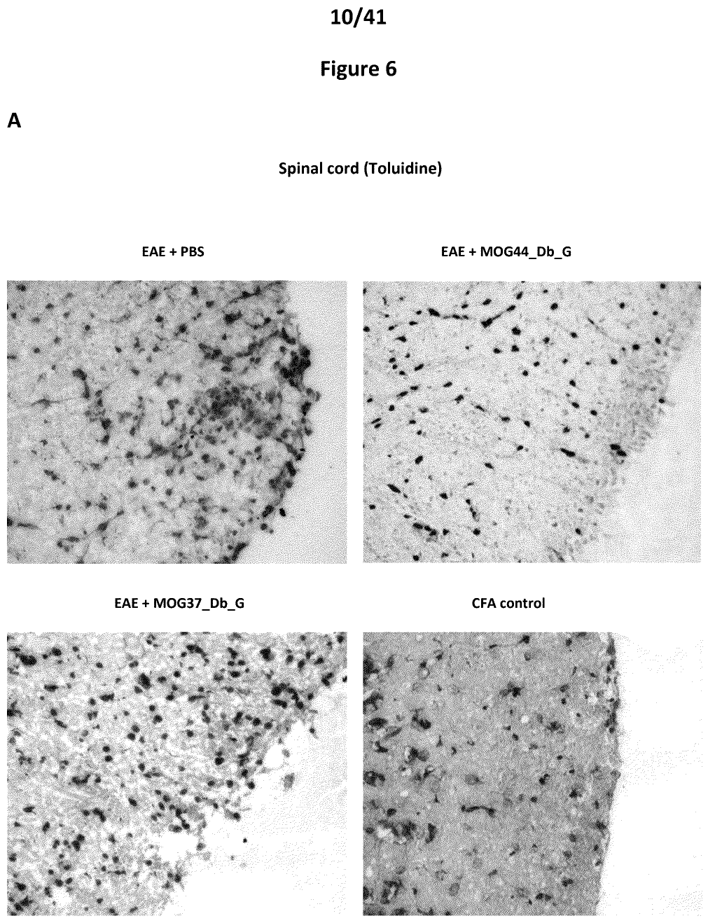

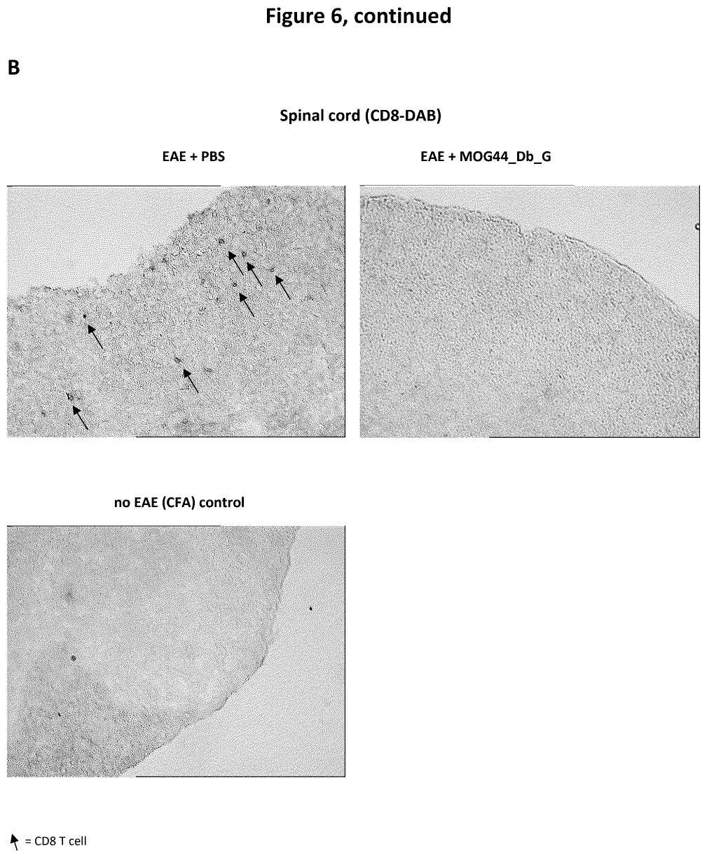

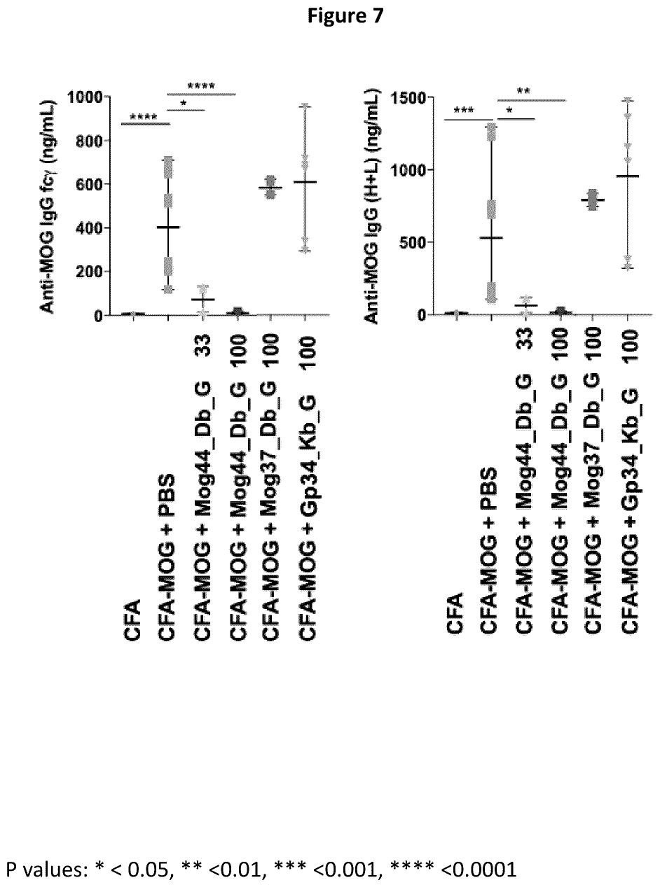

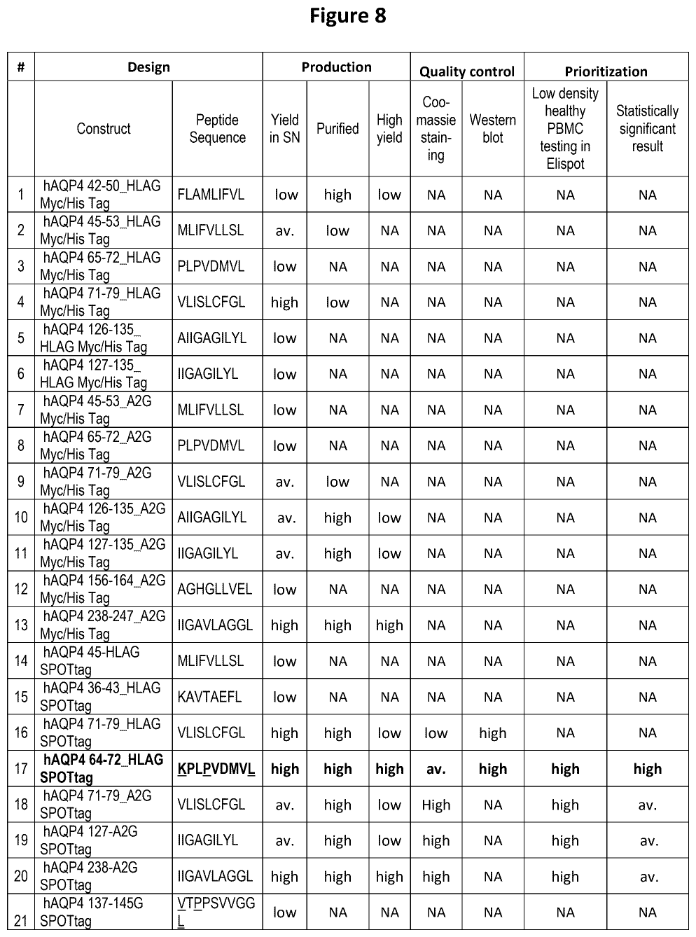

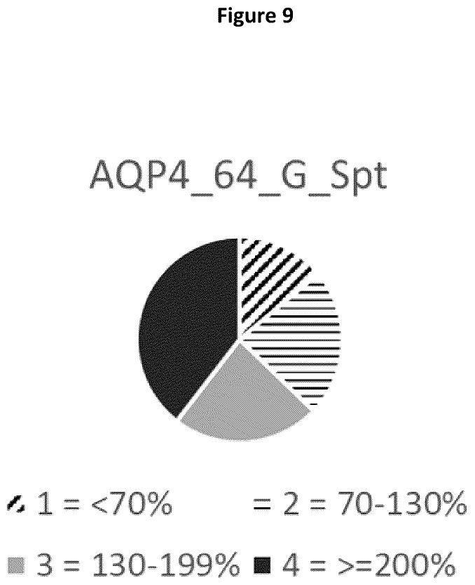

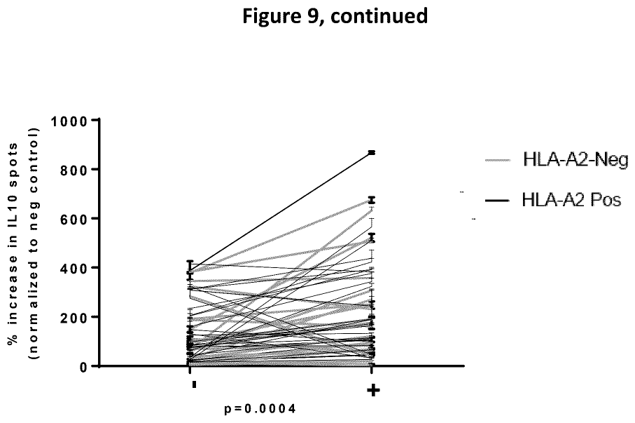

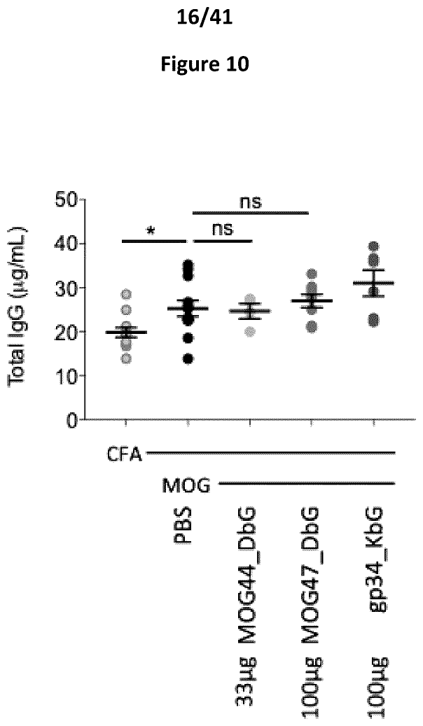

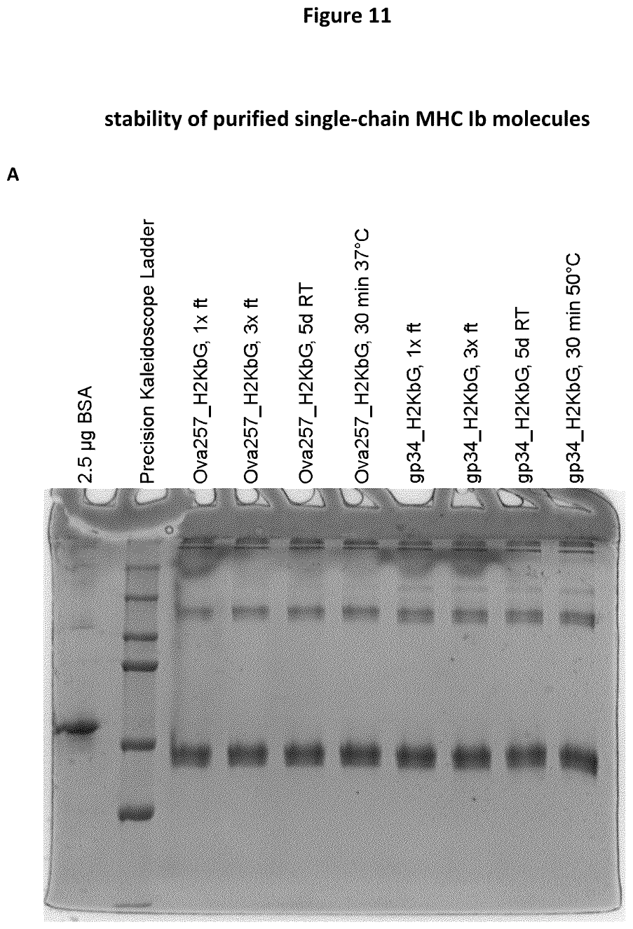

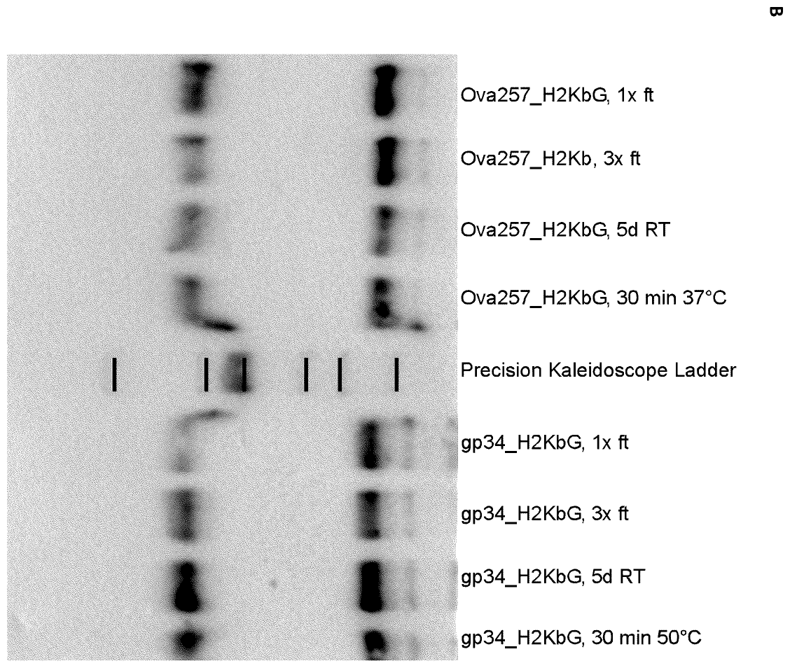

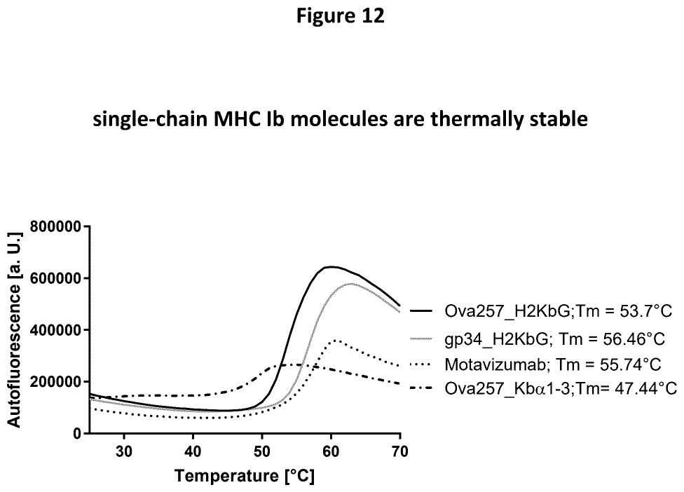

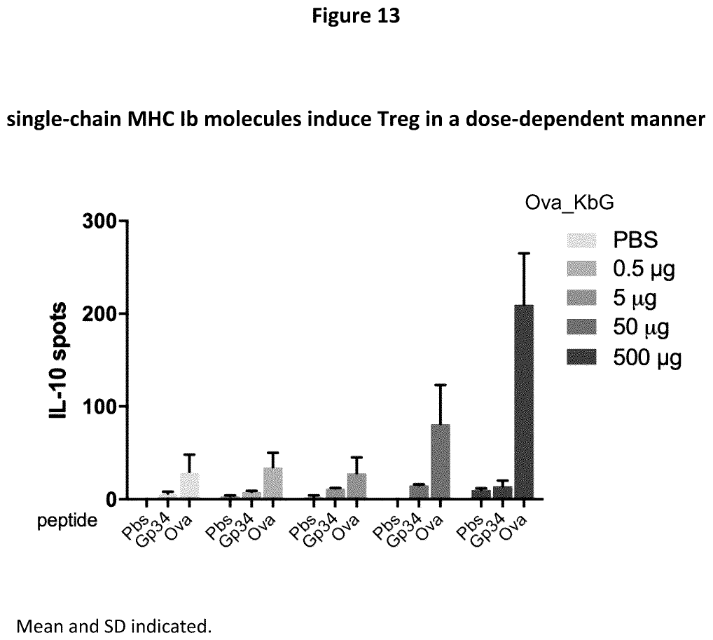

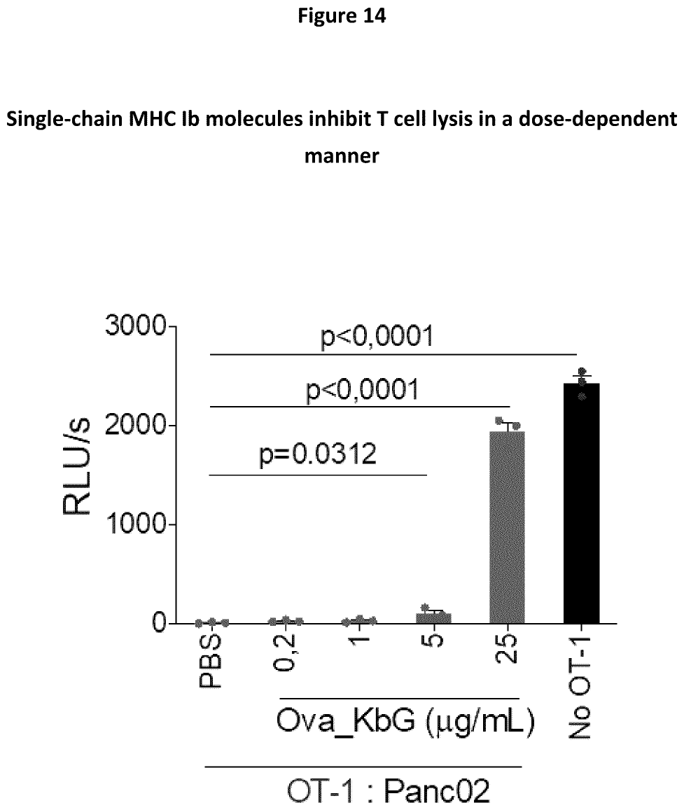

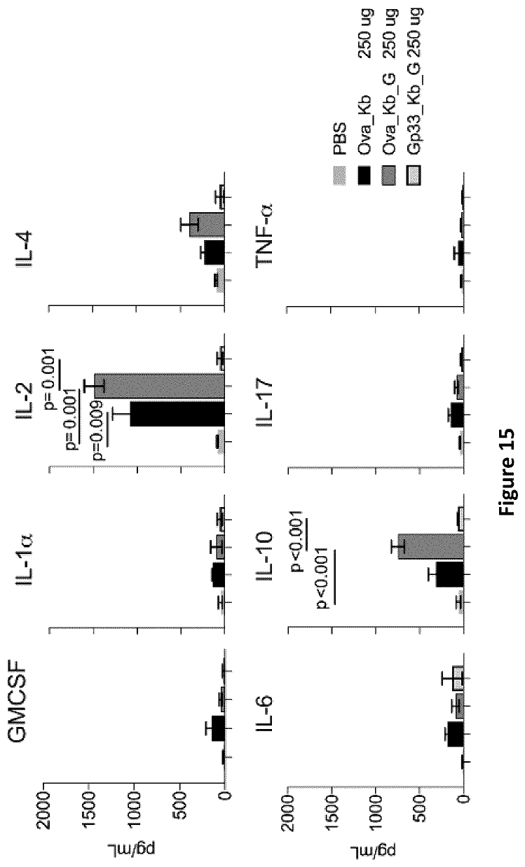

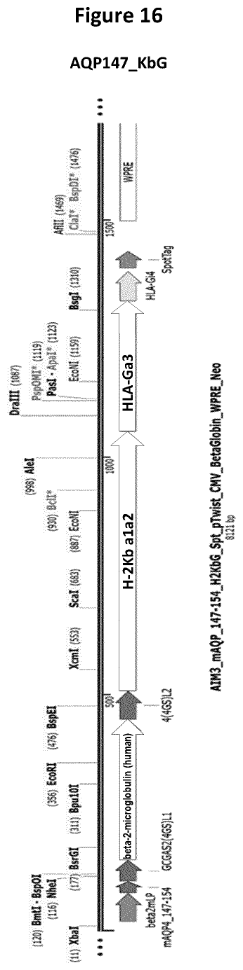

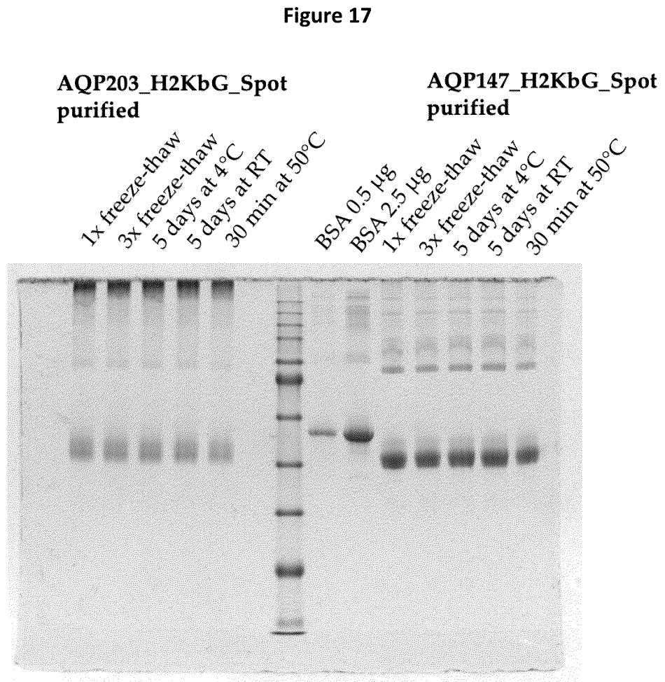

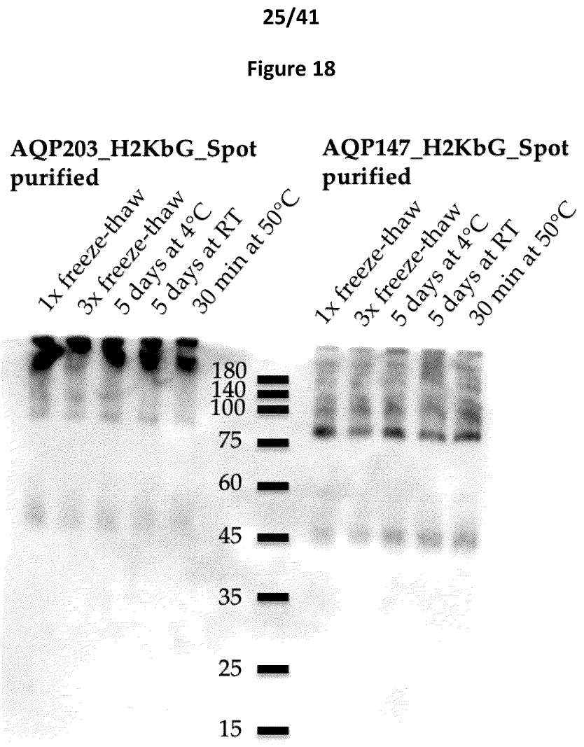

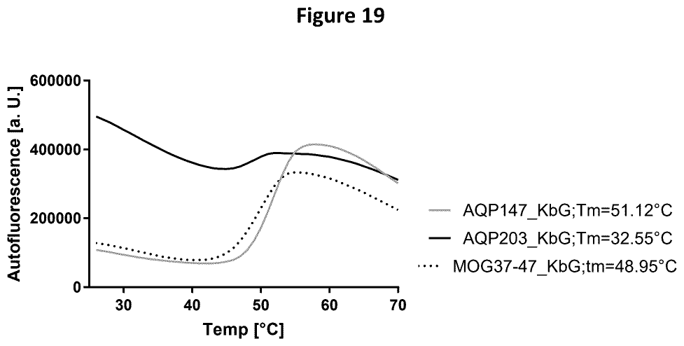

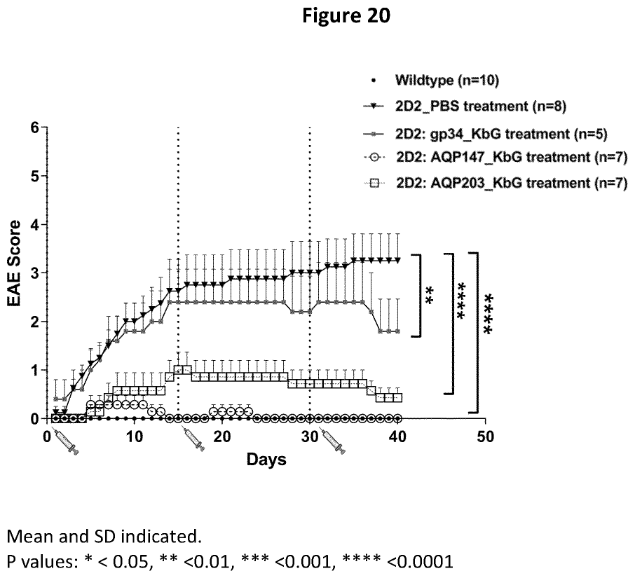

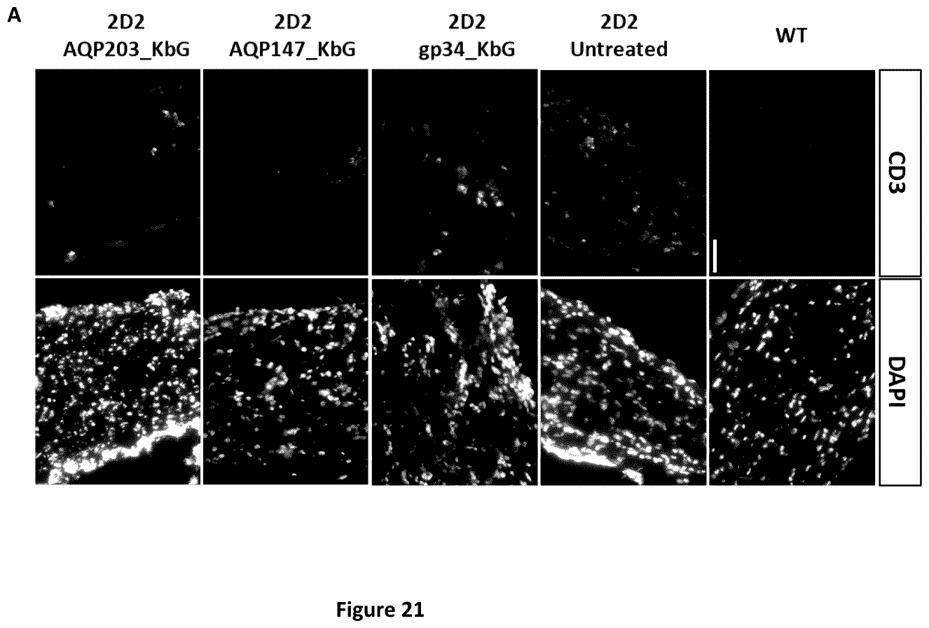

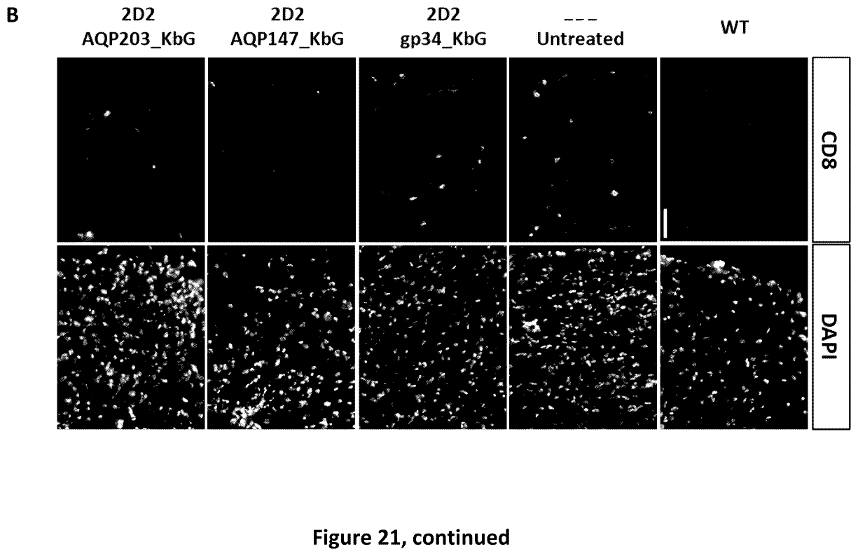

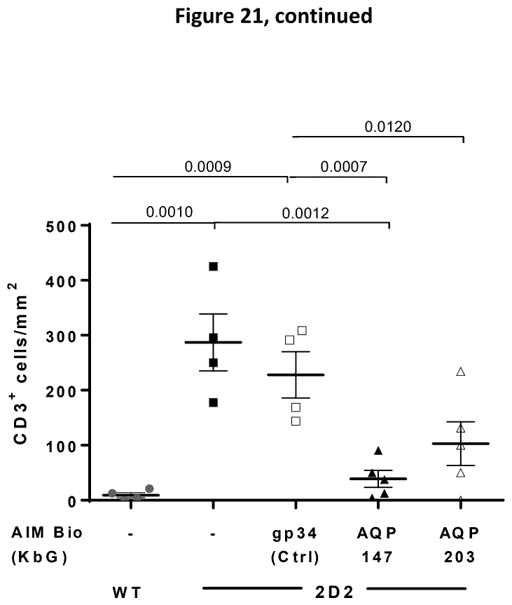

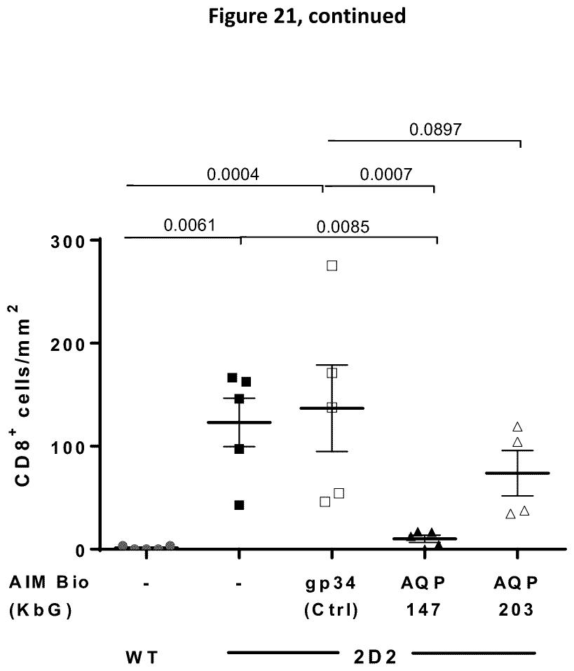

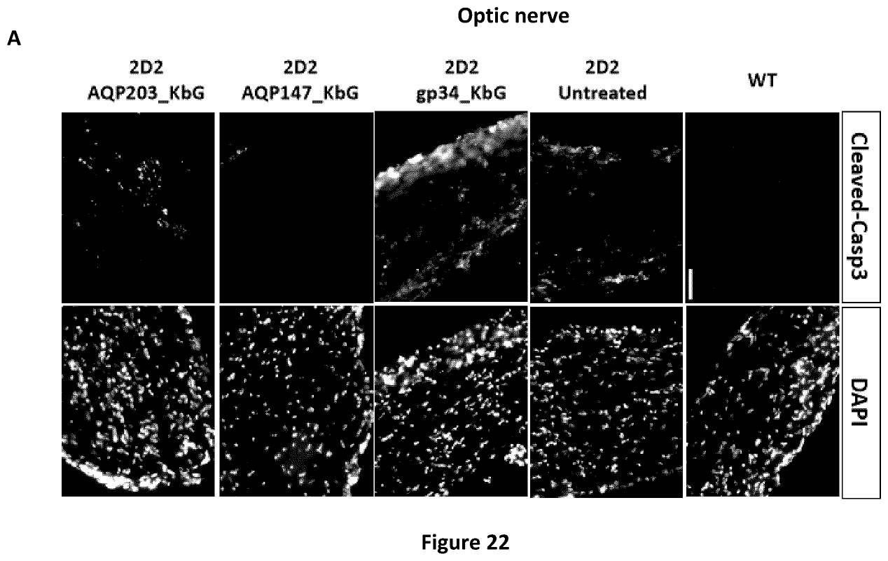

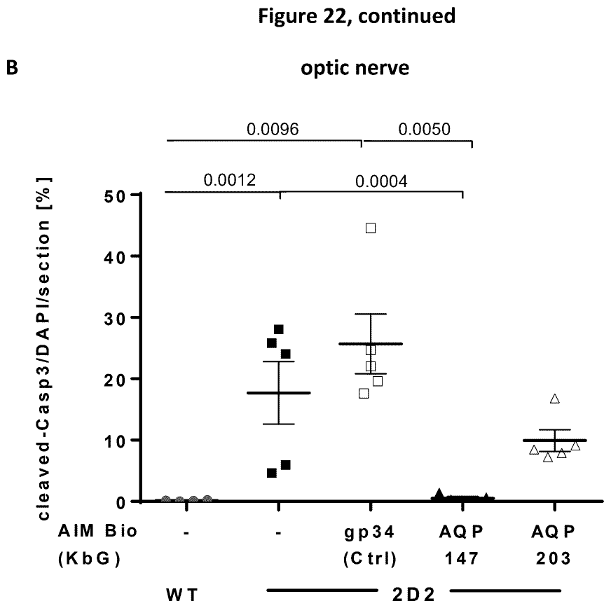

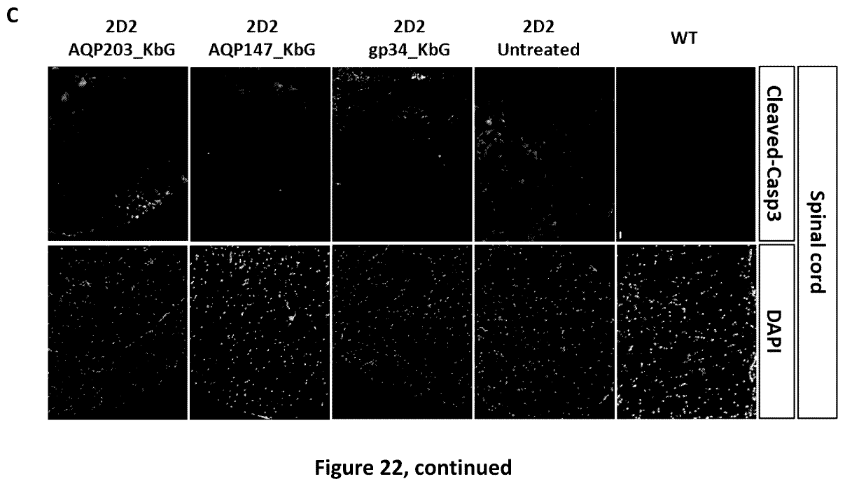

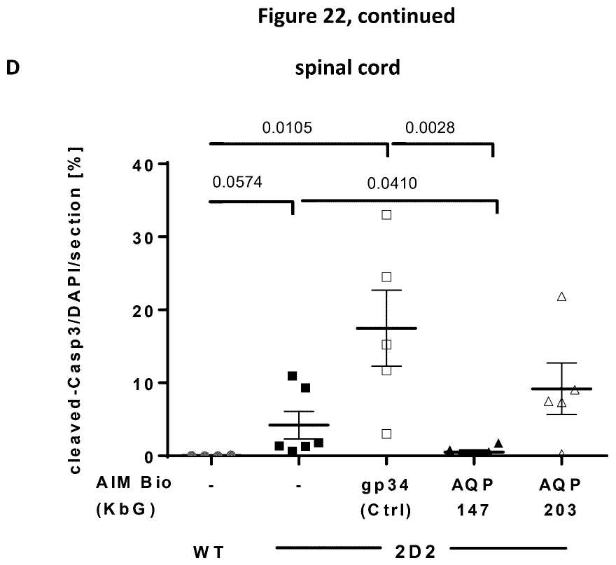

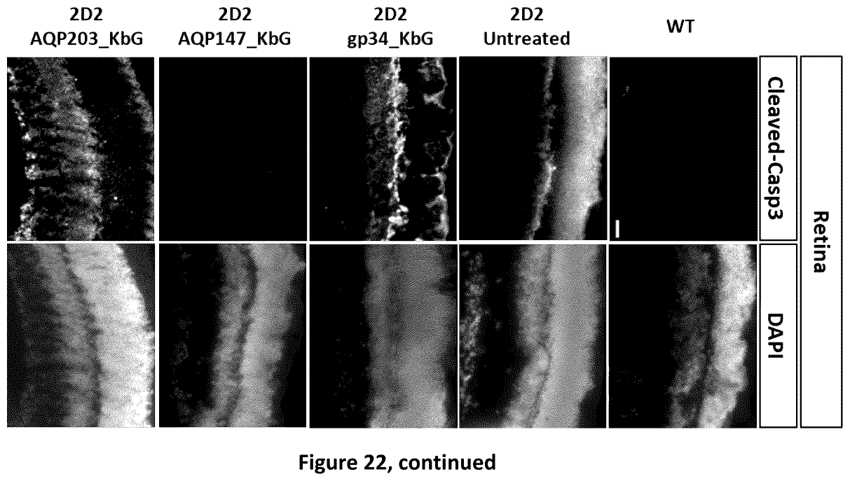

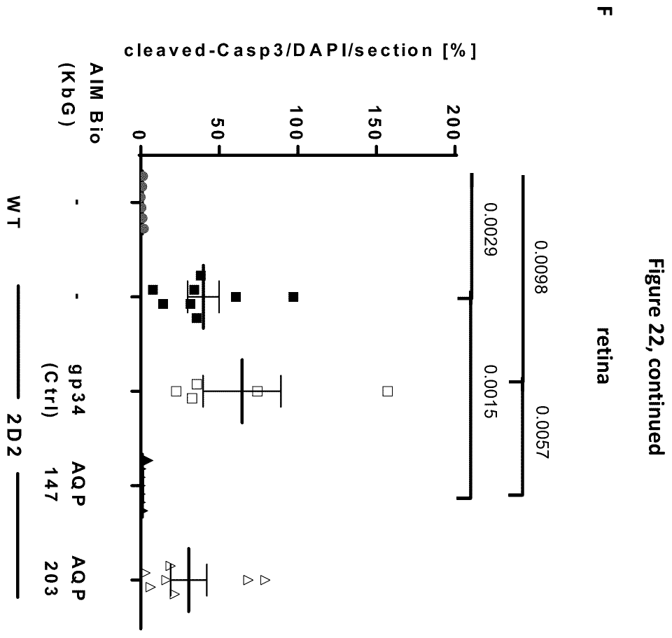

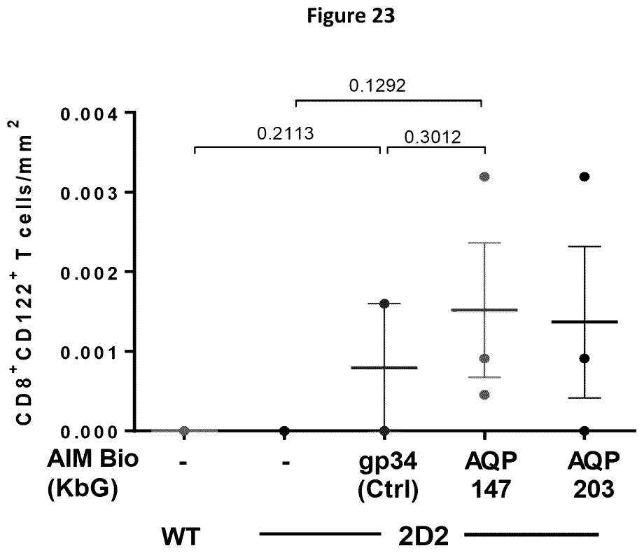

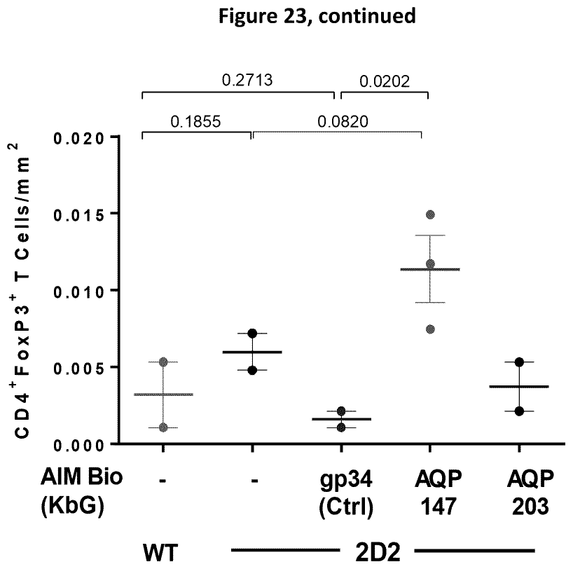

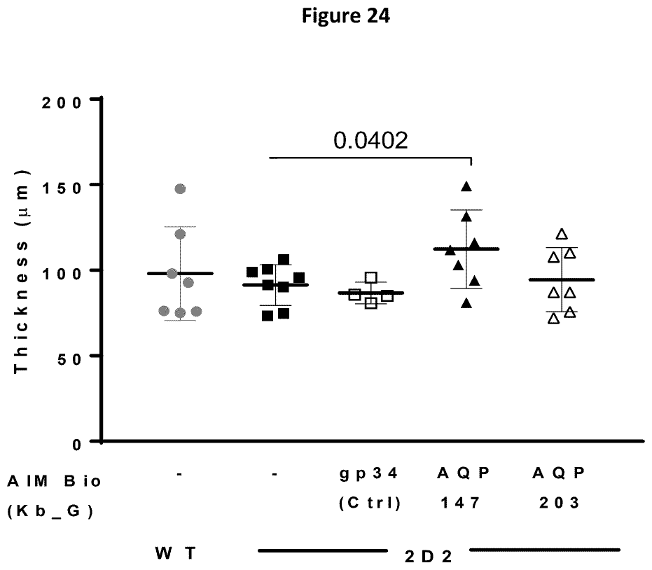

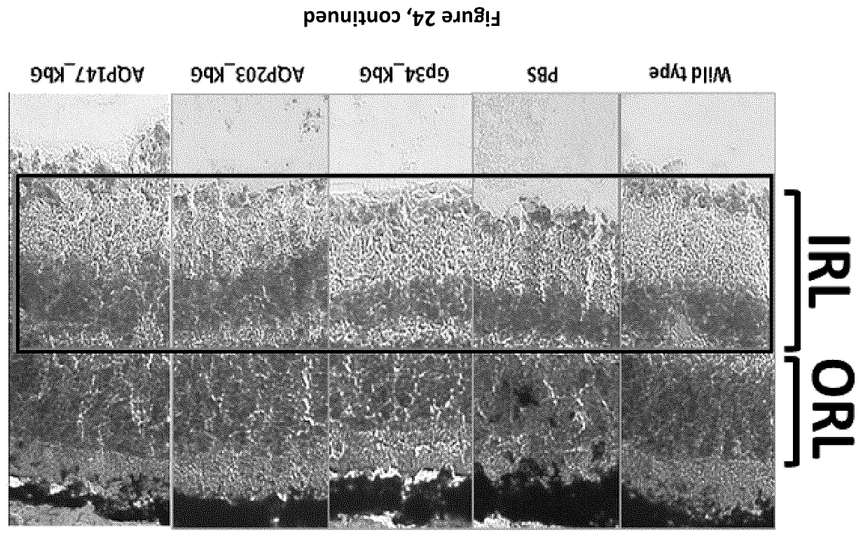

HLA-G1 and HLA-G5 each consist of 3 [alpha] domains (here in black), a non-covalently associated beta 2-microglobulin subunit (here in dark grey) and the antigenic peptide presented on HLA-G (short black arrow). HLA-G1 further contains a transmembrane domain and a short intracellular chain (not shown here). As shown here, the [alpha]-3 domain is capable of binding to the receptors ILT2 (see Shiroishi et al., Proc Natl Acad Sci USA. 2003 Jul. 22; 100(15):8856-8861) and ILT4 (see Shiroishi et al., Proc Natl Acad Sci USA. 2006 Oct. 31; 103(44):16412-7) on immune cells. Physiologically, these sequences form a non-covalently linked MHC class 1 complex. To simplify purification of the complex MHC Ib molecule, one or more protein tags (such as SpotTag, myc tag and/or His(6×) tag) may be introduced. They may be introduced in such a way as to enable their later optional removal via cleavage using an optional Factor Xa cleavage site. Furthermore, the antigenic peptide, beta 2-microglobulin and MHC Ib [alpha]chain can be linked in order to increase the stability. The vector map was generated using Snapgene Viewer Software.

FIG. 3: Surrogates of recombinant polypeptides of the invention induce IL10 secreting Treg in mice.

In this experiment, 100 μg of surrogate molecules consisting of a viral (Gp34) or Ovalbumin (Ova) model peptide antigen, murine H2-K^{b} alpha1 and 2 domains, and human HLA-G alpha 3 domain and beta-2-microglobulin were injected i.p. into 12 week old C57BL/6 mice. After 14 days, mice were sacrificed and splenocytes were rechallenged with 5 μg/ml of either Gp34 or Ova peptide in an 48 h standard murine IL-10 ELIspot assay (Mabtech mouse IL-10 HRP ELISpot kit, cells cultured in RPMI 10% FCS 10 ng/ml IL2).

A significant increase in regulatory T cells that secreted IL-10 only in response to rechallenge with the peptide towards which tolerance was induced via surrogate molecule injection was detectable.

(A) experimental design; (B) results

FIG. 4: Surrogates of recombinant polypeptides of the invention prevent CD8+ T-cell driven EAE in mice.

In this EAE mouse model, the model antigen ovalbumin (OVA) is expressed in oligodendrocytes under the control of the myelin basic protein (MBP) promoter (ODC-OVA). This leads to the presentation of the OVA257-264 peptide on H-2Kb MHC molecules on oligodendrocytes. OT-I mice express a T cell receptor (OT-I) on their CD8+ T cells, which recognizes exactly this peptide-MHC combination. When CD8+ T cells from these mice are transferred into 10 day old ODC-OVA mice, these develop an experimental autoimmune encephalomyelitis (EAE) which resembles in many aspects the pathogenesis and symptomatology of MS (Na et al., Brain, Volume 131, Issue 9, September 2008, Pages 2353-2365). In this experiment, 500 μg of surrogate molecules consisting of a viral (Gp34) or Ovalbumin (Ova) model peptide antigen, murine H2-K^{b} alpha1 and 2 domains, and human HLA-G alpha 3 domain and beta-2-microglobulin or just PBS were injected the same day. EAE was scored according to Bittner et al., J Vis Exp. 2014 Apr. 15; (86):51275.

Only Ovalbumin-tolerance inducing surrogate molecules almost completely prevented EAE symptoms. (A) experimental design; (B) results

FIG. 5: Some surrogates of recombinant polypeptides of the invention selectively prevent CD4⁺ T cell driven EAE in mice.

In this model, a strong, myelin-specific autoimmune response is triggered by administration of MOG 35-55 peptide in combination with Complete Freund's adjuvant, which activates CD4+ Th17 cells, and pertussis toxin, which makes the blood-brain barrier more permeable (Protocol: Bittner et al., J Vis Exp. 2014 Apr. 15; (86):51275). Here, CD4+ cells as well as antibodies play a crucial role in the development of EAE (Tigno-Aranjuez et al., J Immunol Nov. 1, 2009, 183 (9) 5654-5661). In addition, 100 μg/mouse of surrogate molecules consisting of a viral (Gp34) or two Mog peptide antigens (Mog37 or Mog44), murine H2-D^{b} alpha1 and 2 domains, and human HLA-G alpha 3 domain and beta-2-microglobulin or just PBS were injected the first day.

The Mog44 peptide containing surrogate molecule significantly reduced EAE symptoms and weight loss.

(A) experimental design; (B) EAE score; (C) body weight

FIG. 6: Mog44 surrogates of recombinant polypeptides of the invention prevented inflammation and CD8 T cell infiltration in the spinal cord.

10 μm fresh frozen sections were stained with commercial Toluidine 1× staining reagent for 1 h at room temperature. A strong infiltration of immune cells was detected in EAE, but prevented by Mog44_Db_G.

10 μm fresh frozen sections were briefly dried at room temperature, fixed with acetone, blocked with 5% BSA 10% normal goat serum in PBS, stained with 1:100 anti-CD8 antibody, secondary antibody coupled to HRP and DAB solution (detailed methods: Karikari et al., Brain Behav Immun. 2022 Jan. 12; 101:194-210). Mog35-55 induced EAE lead to a strong infiltration of CD8⁺ cells into the spinal cord which was prevented by MOG44_Db_G surrogate molecule.

(A) Toluidine; (B) CD8-DAB

FIG. 7: Detection of anti-MOG35-55 antibodies in Mog-EAE mice treated with surrogates of recombinant polypeptides of the invention (“AIM Bio”)

Murine serum was collected from heart puncture after mice were sacrificed. 10 μg/ml Mog35-55 were used for coating over night, wells were blocked using 1% BSA, and anti-Mog35-55 antibodies were detected using the indicated secondary HRP coupled antibodies.

Mog35-55 induced EAE correlated with high levels of Mog35-55 specific IgG autoantibodies, which were not detectable in animals treated with 100 μg MOG44_Db_G surrogate molecule.

FIG. 8: List of the human NMO recombinant polypeptide candidates.

The peptide antigen sequences of the NMO recombinant polypeptide candidates shown in the Figure are as follows:

| | Peptide | |
|---|---|---|
| Construct | antigen | SEQ ID NO: |
| hAQP4 42-50_HLAG Myc/His Tag | FLAMLIFVL | SEQ ID NO: 31 |
| hAQP4 45-53_HLAG Myc/His Tag | MLIFVLLSL | SEQ ID NO: 32 |
| hAQP4 65-72_HLAG Myc/His Tag | PLPVDMVL | SEQ ID NO: 33 |
| hAQP4 71-79_HLAG Myc/His Tag | VLISLCFGL | SEQ ID NO: 22 |
| hAQP4 126-135_HLAG Myc/His Tag | AIIGAGILYL | SEQ ID NO: 34 |
| hAQP4 127-135_HLAG Myc/His Tag | IIGAGILYL | SEQ ID NO: 23 |
| hAQP4 45-53_A2G Myc/His Tag | MLIFVLLSL | SEQ ID NO: 32 |
| hAQP4 65-72_A2G Myc/His Tag | PLPVDMVL | SEQ ID NO: 33 |
| hAQP4 71-79_A2G Myc/His Tag | VLISLCFGL | SEQ ID NO: 22 |
| hAQP4 126-135_A2G Myc/His Tag | AIIGAGILYL | SEQ ID NO: 34 |
| hAQP4 127-135_A2G Myc/His Tag | IIGAGILYL | SEQ ID NO: 23 |
| hAQP4 156-164_A2G Myc/His Tag | AGHGLLVEL | SEQ ID NO: 35 |
| hAQP4 238-247_A2G Myc/His Tag | IIGAVLAGGL | SEQ ID NO: 24 |
| hAQP4 45-HLAG SPOTtag | MLIFVLLSL | SEQ ID NO: 32 |
| hAQP4 36-43_HLAG SPOTtag | KAVTAEFL | SEQ ID NO: 36 |
| hAQP4 71-79_HLAG SPOTtag | VLISLCFGL | SEQ ID NO: 22 |
| hAQP4 64-72_HLAG SPOTtag | KPLPVDMVL | SEQ ID NO: 2 |
| hAQP4 71-79_A2G SPOTtag | VLISLCFGL | SEQ ID NO: 22 |
| hAQP4 127-A2G SPOTtag | IIGAGILYL | SEQ ID NO: 23 |
| hAQP4 238-A2G SPOTtag | IIGAVLAGGL | SEQ ID NO: 24 |
| hAQP4 137-145_HLAG SPOTtag | VTPPSVVGGL | SEQ ID NO: 37 |

FIG. 8 further shows which combinations of AQP4 peptides and antigen-presenting MHC class I alpha 1 and 2 domains (HLAg=HLA-G, A2G=HLA-A2 presenting domains+HLA-G alpha 3 domain) lead to successful (high) intermediate (average, av.) or non-preferable (low) results with regards to production, quality control and prioritization in healthy blood donors (described in FIG. 9)

FIG. 9: Upregulation of CD8 Treg in healthy blood donors by a recombinant polypeptide of the invention containing the KPLPVDMVL antigen (“AQP_64”)

In vitro Treg induction mediated by peptide-HLA-G containing constructs (AIM Biologicals) was carried out as follows: PBMCs from healthy donors were purified via density centrifugation performed on white blood cells from a leukocyte reduction chamber using Ficoll. Cells were centrifuged for 20 min at 1200×g without brake followed by collection of the interphase ring that was washed with 1×PBS (5 min, 300×g). PBMC were frozen till further use.

PBMCs were thawed 1 day prior to PBMC pulsing (d-1) and kept over night in 5 ml X-VIVO 15 medium containing 5% human AB serum in a well of a 6 well plate at 37° C.

On the next day (d0), cells were counted and resuspended in X-VIVO 15 complete medium (5% hAB serum & cytokine cocktail: 20 ng/ml hIL-2, 20 ng/ml hGM-CSF, 10 ng/ml hIL-4 & 10 ng/ml hTGF-b1) at a cell density of 3×10⁶ cells/ml. For experiments, 3×10⁶ cells were seeded in the respective wells of a 12-well plate with a final volume of 1000 μl X-VIVO complete medium with cytokine cocktail and 5 μg/ml of an AIM Bio molecule or the respective controls.

On day 3, 1 ml complete medium (with cytokines) was added, on day 6, a second pulse with 5 μg/ml AIM Bio molecule was performed (after removing medium). On days 7, 10 & 12, 1 ml complete medium (with cytokines) was added.

On day 13, ELISpot plate PVDF membrane was activated with 50 μl/well EtOH (35% v/v) for 1 min followed by 5× washing with 200 μl distilled sterile water. Plate was coated with 100 μl/well anti-hIL10 (clone 9D-7, 1:500 dilution in PBS, sterile filtered) at 4° C. over night. On the next day, unbound coating antibody was removed, 5 washing steps were performed with 200 μl PBS and 200 μl blocking buffer (X-VIVO 15 5% hAB serum) was added and the plate incubated for 30 min-2 h at room temperature. Day 14, 200,000 cells were seeded per well on the ELISpot plates in duplicates, including negative controls (cells plus PBS) and a positive control (e.g. LPS) for 48 h. Secondary antibody was prepared: 1 μg/ml aIL-10-biotinylated antibody in 0.5% BSA/1×PBS (1:1000 dilution) and horseradish peroxidase-conjugated streptavidin (1:750 in 0.5% BSA/PBS), tetramethylbenzidine solution was filtered using a 0.45 μm filter and stored at 4° C. till use. Cell supernatant was removed and 5× washed using 100 μl PBS. Last excess buffer was removed using paper towels. 25 μl diluted HRP-streptavidin (1:750) was added per well and incubated for 1 h at room temperature in the dark followed by 5 washing steps using sterile 1×PBS. 100 μl of filtered TMB substrate was added per well for 15-25 min till blue sports developed. Reaction was stopped by washing the wells thoroughly with water. Plastic underdrains of the plates were removed and the bottom and sides of the plates were also washed with tap water and dried.

AQP4_64_G_Spt induced at least 30% more IL-10 secreting T reg in 65% of all healthy blood donors.

FIG. 9, continued: % increase in IL10 spots in PBMCs shown for HLA-A2⁺ abd HLA-A2⁻ donors (in response to treatment with AQP4_64_G_Spt).

FIG. 10: Control experiment on the samples shown in FIG. 7 shows that total IgG is not reduced by MOG47_Db_G surrogate molecule treatment. Easy-Titer™ Human IgG (gamma chain) Assay Kit (Thermo Fisher) was used to quantify total IgG according to the manufacturers instructions. This experiments in conjunction with FIG. 7 indicates that selective antibody responses can be suppressed using single-chain MHC Ib molecules.

FIG. 11: Stability of purified single-chain MHC Ib molecules. After purification of the single chain MHC Ib molecules, their stability was analysed after 1 and 3 freeze-thawing cycles, storage for 5 days at room temperature and heating up to a temperature of 50° C. for 30 min. For this, A) a Coomassie gel staining of a 12% polyacrylamide gel using 2 μg AIM Bio and B) an aHLA-G Western blot using the 2A12aHLA-G antibody (1:1000) blot using 1 μg protein was performed under non-reducing conditions. Both monomers and dimers are detectable.

FIG. 12: Single-chain MHC Ib molecules are thermally stable. For the Thermal Shift Assay (TSA), 3 μg of the respective single chain MHC Ib molecule or Motavizumab as control molecule were diluted with PBS and 5×SYPRO Orange dye (stock 5000×, final concentration: 5×) to a volume of 25 μl. A melting curve program was set up on a StepOnePlus Instrument using the StepOnePlus Software 2.3. The start temperature was 25° C. for one minute followed by a temperature increase of 1° C. per minute to a final temperature of 95° C. for 2 min, thereby measuring the autofluorescence as arbitrary unit. Data were exported and graphs were drawn in Prism V7.04. For determination of the melting temperature (Tm), the Boltzman sigmoidal function was used.

FIG. 13: Single-chain MHC Ib molecules induce Treg in a dose-dependent manner. OT-I mice were injected i.p. with indicated amounts of single-chain H2_K^{b} alpha1+2 and HLA-G alpha3 domain constructs with human beta-2-microglobulin and the indicated peptide or carrier (PBS). Ova is the cognate peptide for the OT-I TCR in these mice, Gp34 is an irrelevant, virus derived control peptide. After 14 days, mice were sacrificed and splenocytes tested for IL10 secreting cells in a recall mouse IL-10 ELISpot (200,000 cells per well, MabTech mouse IL-10 ELISpot kit, 5 μg/ml of the indicated peptide or only PBS were added, 48 h). A clear induction of IL-10 secreting cells reactive to Ova peptide was observed when 50 and 500 μg mouse adapted Ova_KbG were injected.

FIG. 14: Single-chain MHC Ib molecules inhibit T cell lysis in a dose-dependent manner. OT1/BL6 Mice were sacrificed and splenocytes were collected and washed once in RPMI 5% FCS. Red blood cells were removed with 2 ml 1× sterile RBC lysis buffer for 3 min. Cells were cultured in high density culture (10mio cells/ml) for 72 h in RPMI 10% FCS medium with GMCSF 20 ng/mL, IL-2 20 ng/ml and IL-4 10 ng/ml and increasing doses of Ova_KbG. Cells are then scraped from the plates, CD8+ cells are then purified via magnetic beads. Sterile 96-well white plates were used. Luciferase expressing Panc02 target cells were loaded with 20 μg/ml Ova peptide (SIINFEKL) for 60 min at 37° C. with 500 rpm shaking. CD8+ effector T cells were added in a 50:1 ratio, as well as luciferin. Luminescence was measured after 24 h and 48 h.

FIG. 15: Serum cytokines from EAE-ODC Ova mice. Serum cytokines from EAE-ODC Ova mice were measured with Th1/Th2 10plex Flowcytomix Kit (eBioscience) according to the manufacturer's instruction. The kit was used for the simultaneous detection of mouse granulocyte-macrophage colony-stimulating factor (GMCSF), interleukin 1 alpha (IL-1a), interleukin-2 (IL-2), interleukin-4 (IL-4), interleukin-6 (IL-6), interleukin-10 (IL-10), t interleukin-17 (IL-17), and tumor necrosis factor (TNF) in a single sample. This array kit provides a mixture of eight capture beads with distinct fluorescent intensities that have been coated with capture antibodies specific for each cytokine. Beads coated with ten specific capture antibodies were mixed. Subsequently, 25 μL of the mixed captured beads, 25 μL of the unknown serum sample or standard dilutions, and 25 μL of phycoerythrin (PE) detection reagent were added consecutively to each well in 96-V bottom well plates and incubated for 2 h at room temperature in the dark. The samples were washed with 1 mL of wash buffer for 5 min and centrifuged. The bead pellet was resuspended in 200 μL buffer after discarding the supernatant. Samples were measured on the Attune™ N×T Flow Cytometer and analyzed Attune Cytometric Software (Thermo Fisher Scientific).

FIG. 16: Map of molecular composition of most-preferred mouse-adapted AIM Bio.

A corresponding exemplary amino acid sequence is:

(SEQ ID NO: 38)
MSRSVALAVLALLSLSGLEAVTTVHGNLGCGASGGGGSGGGGSIQ
RTPKIQVYSRHPAENGKSNFLNCYVSGFHPSDIEVDLLKNGERIE
KVEHSDLSFSKDWSFYLLYYTEFTPTEKDEYACRVNHVTLSQPKI
VKWDRDMGGGGSGGGGSGGGGSGGGGSGPHSLRYFVTAVSRPGLG
EPRYMEVGYVDDTEFVRFDSDAENPRYEPRARWMEQEGPEYWERE
TQKAKGNEQSFRVDLRTLLGCYNQSKGGSHTIQVISGCEVGSDGR
LLRGYQQYAYDGCDYIALNEDLKTWTAADMAALITKHKWEQAGEA
ERLRAYLEGTCVEWLRRYLKNGNATLLRTDPPKTHVTHHPVFDYE
ATLRCWALGFYPAEIILTWQRDGEDQTQDVELVETRPAGDGTFQK
WAAVVVPSGEEQRYTCHVQHEGLPEPLMLRWSKEGDGGIMSVRES
RSLSEDLGSPDRVRAVSHWSSC

FIG. 17: Coomassie gel of purified, stability tested NMO surrogate single-chain MHC Ib molecules. After purification of the single chain MHC Ib molecules, their stability was analysed after 1 and 3 freeze-thawing cycles, storage for 5 days at room temperature and heating up to a temperature of 50° C. for 30 min. Methods see FIG. 11.

FIG. 18: Western blot (2A12 HLA-G5 antibody 1:5000) of purified and stability tested NMO surrogate single-chain MHC Ib molecules. Both experiments indicated that these molecules are very stable, but AQP203_H2KbG tends to multimerize much more.

FIG. 19: Thermal Shift Assay (TSA). The results obtained in the preceding Figure were also confirmed in a TSA, in which multimers result in high autofluorescence at low temperatures.

FIG. 20: EAE Score. AQP147_KbG conferred complete protection from EAE in treated 2D2 mice. Mice were injected with treatment or control AIM Bios on DO, D15 and D30. They were evaluated daily based on EAE scoring system of 0-10 described in table 1. Untreated mice developed severe EAE symptoms during the course of experiment. 1 untreated mouse reached a score of 6 on D14 and was sacrificed. Its score was assumed as 6 for the remaining days during evaluation. Control treatment with gp34_KbG did not confer desirable protection. (*p<0.05, **p<0.01, ***p<0.001)

FIG. 21: Immune cell infiltration in optic nerve of wildtype and 2D2 mice. AQP147_KbG treatment inhibited immune cell infiltration in optic nerve. CD3⁺ and CD8⁺ fluorescence signals obtained from each treatment group are shown in panels A (optic nerve) and B, (spinal cord) DAPI images depict total nuclei present in each section. The number of CD3⁺ and CD8⁺ T cells were significantly higher in the optic nerve (panel C) and spinal cord (panel D) of control and untreated mice. AQP203_KbG inhibited immune infiltration better than control and untreated groups. Data is represented as mean+/−SEM. Scale bar 25 μm. 1-tailed t-test.

FIG. 22: Cleaved-Caspase-3 quantification in optic nerve, spinal cord and retina of wildtype and 2D2 mice. AQP147_KbG completely inhibits apoptosis in optic nerve, spinal cord and retina. Fluorescent signals obtained from cleaved-caspase-3 (apoptosis marker) staining in optic nerve (panels A, B) spinal cord (panels C, D) and retina (panels E, F) are shown. Untreated and control treated mice had highest levels of cleaved-casp-3 and thus in turn, highest level of apoptosis. Data is represented as mean+/−SEM. Scale bar 25 μm. 1-tailed t-test

FIG. 23: CD8⁺CD122⁺ and CD4⁺FoxP3⁺ regulatory T cells in optic nerve. The Figure shows that while there are less CD8 T cells in AIM treated mice, there are few, but clearly more regulatory T cells in the optic nerve (one-tailed T Test used)

FIG. 24: Quantification of inner retinal thickness in wildtype and 2D2 mice. AQP147_KbG completely rescues IRL cells from degradation. Hematoxylin-eosin staining results show that the thickness of inner retinal layer of AQP147_KbG was preserved whereas untreated and control treated mice had thinner IRL. Data is represented as mean+/−SEM. Scale bar 25 μm. 2-tailed t-test

### DETAILED DESCRIPTION OF THE INVENTION

### Definitions and General Techniques

Unless otherwise defined below, the terms used in the present invention shall be understood in accordance with their common meaning known to the person skilled in the art. All publications, patents and patent applications cited herein are hereby incorporated by reference in their entirety for all purposes. Publications referred to herein may be cited by specifying the full literature reference in the text.

All proteins in accordance with the invention, including the recombinant polypeptides of the invention, can be obtained by methods known in the art. Such methods include methods for the production of recombinant polypeptides. The recombinant polypeptides of the invention can be expressed in recombinant host cells according to the invention. Recombinant host cells of the invention are preferably mammalian cells such as CHO and HEK cells.

It will be understood that the recombinant polypeptides of the invention are meant to optionally include a secretion signal peptide sequence. Similarly, the recombinant polypeptides of the invention are meant to also optionally include affinity tags, e.g. in order to facilitate purification, and optional protease cleavage sites between the tag and the polypeptide, e.g. in order to facilitate removal of the tags by protease cleavage.

It is also understood that any reference to amino acid sequences referred to herein is meant to encompass not only the unmodified amino acid sequence but also typical posttranslational modifications of these amino acid sequences (e.g., glycosylation or deamidation of amino acids, the clipping of particular amino acids or other posttranslational modifications) occurring in cellular expression systems known in the art, including mammalian cells such as CHO and HEK cells.

Likewise, it will be understood that the recombinant polypeptides of the invention are meant to optionally include the respective pro-peptides.

It will also be understood that the recombinant polypeptides of the invention can be in form of their soluble or their membrane-bound form. Whether a recombinant polypeptide is “soluble” under these conditions can be determined by methods known in the art, e.g., by measuring the turbidity of the recombinant polypeptide under the above-indicated reference conditions. As used herein, soluble means that at least 95% of the recombinant polypeptide is determined to be soluble under these reference conditions.

Single chain MHC molecules can be stored, for instance, in PBS at −80° C. (with or without 0.1% human albumin as carrier, depending on the protein concentration) or in 50% glycerol at −20° C.

According to the invention, MHC molecules are preferably human MHC molecules.

The recombinant polypeptides of the invention are preferably isolated recombinant polypeptides.

It will be understood how a recombinant polypeptide capable of binding and presenting an peptide antigen according to the invention can be prepared. For example, peptide antigen-binding domains such as [alpha]1 and [alpha]2 domains are well-known, and modifications of these domains can be made. The capability of a peptide antigen to bind to the polypeptides and MHC molecules according to the invention can be determined by techniques known in the art, including but not limited to explorative methods such as MHC peptide elution followed by Mass spectrometry and bio-informatic prediction in silico, and confirmative methods such as MHC peptide multimere binding methods and stimulation assays.

In accordance with the invention, the recombinant polypeptides, pharmaceutical compositions and kits of the invention are preferably suitable for use in a human patient.

In accordance with the invention, the recombinant polypeptides, pharmaceutical compositions and kits of the invention are preferably suitable for use in the treatment of neuromyelitis optica in a human patient.

In accordance with the invention, the recombinant polypeptides, pharmaceutical compositions and kits of the invention are preferably suitable for inducing immunological tolerance against human aquaporin 4, e.g., in a human patient.

It is understood that in accordance with the invention, the recombinant polypeptides, pharmaceutical compositions and kits of the invention are stable.

It will be understood that in connection with the peptide antigens used in accordance with the invention, any lengths of these peptide antigens referred to herein (e.g. “7 to 11 amino acids in length”) are meant to refer to the length of the peptide antigens themselves. Thus, the lengths of peptide antigens referred to herein do not include the length conferred by additional amino acids which are not part of the peptide antigens such as additional amino acids from possible linker sequences etc.

In accordance with the present invention, each occurrence of the term “comprising” may optionally be substituted with the term “consisting of”.

### Methods and Techniques

Generally, unless otherwise defined herein, the methods used in the present invention (e.g. cloning methods or methods relating to antibodies) are performed in accordance with procedures known in the art, e.g. the procedures described in Sambrook et al. (“Molecular Cloning: A Laboratory Manual.”, 2^{nd} Ed., Cold Spring Harbor Laboratory Press, Cold Spring Harbor, New York 1989), Ausubel et al. (“Current Protocols in Molecular Biology.” Greene Publishing Associates and Wiley Interscience; New York 1992), and Harlow and Lane (“Antibodies: A Laboratory Manual” Cold Spring Harbor Laboratory Press, Cold Spring Harbor, New York 1988), all of which are incorporated herein by reference.

Protein-protein binding, such as binding of antibodies to their respective target proteins, can be assessed by methods known in the art. Protein-protein binding is preferably assessed by surface plasmon resonance spectroscopy measurements.

For instance, binding of MHC class Ib molecules or recombinant polypeptides according to the invention to their receptors, including ILT2 and ILT4, is preferably assessed by surface plasmon resonance spectroscopy measurements. More preferably, binding of MHC class Ib molecules or recombinant polypeptides according to the invention to their receptors is assessed by surface plasmon resonance measurements at 25° C. Appropriate conditions for such surface plasmon resonance measurements have been described by Shiroishi et al., Proc Natl Acad Sci USA. 2003 Jul. 22; 100(15):8856-8861.

Sequence Alignments of sequences according to the invention are performed by using the BLAST algorithm (see Altschul et al. (1990) “Basic local alignment search tool.” Journal of Molecular Biology 215. p. 403-410; Altschul et al.: (1997) Gapped BLAST and PSI-BLAST: a new generation of protein database search programs. Nucleic Acids Res. 25:3389-3402.). Appropriate parameters for sequence alignments of short peptides by the BLAST algorithm, which are suitable for peptide antigens in accordance with the invention, are known in the art. Most software tools using the BLAST algorithm automatically adjust the parameters for sequence alignments for a short input sequence. In one embodiment, the following parameters are used: Max target sequences 10; Word size 3; BLOSUM 62 matrix; gap costs: existence 11, extension 1; conditional compositional score matrix adjustment. Thus, when used in connection with sequences, terms such as “identity” or “identical” preferably refer to the identity value obtained by using the BLAST algorithm.

### Preparation of Pharmaceutical Compositions of the Invention

Pharmaceutical compositions of the present invention are prepared in accordance with known standards for the preparation of pharmaceutical compositions.

For instance, the pharmaceutical compositions are prepared in a way that they can be stored and administered appropriately. The pharmaceutical compositions of the invention may therefore comprise pharmaceutically acceptable components such as carriers, excipients and/or stabilizers.

Such pharmaceutically acceptable components are not toxic in the amounts used when administering the pharmaceutical composition to a human patient. The pharmaceutical acceptable components added to the pharmaceutical compositions may depend on the chemical nature of the active ingredients present in the composition, the particular intended use of the pharmaceutical compositions and the route of administration. In general, the pharmaceutically acceptable components used in connection with the present invention are used in accordance with knowledge available in the art, e.g. from Remington's Pharmaceutical Sciences, Ed. A R Gennaro, 20th edition, 2000, Williams & Wilkins, PA, USA. Pharmaceutical compositions comprising the nucleic acids of the invention (e.g., RNAs) may also be formulated in accordance with knowledge available in the art, e.g. using liposomal formulations targeting dendritic cells.

Peptide Antigens in Accordance with the Invention

The peptide antigens which can be used in accordance with the invention, including the peptide antigens as defined above, are not particularly limited other than by their ability to be presented on MHC molecules. It is understood that a “peptide antigen presented by said recombinant polypeptide” as referred to in relation to the invention is a peptide antigen that is presented by said recombinant polypeptide to human T cells, if such T cells are present, in a way that it binds to a T cell receptor on the human T-cells.

Peptides which are able to be presented on MHC molecules can be generated as known in the art (see, for instance, Rammensee, Bachmann, Emmerich, Bachor, Stevanović, SYFPEITHI: database for MHC ligands and peptide motifs. Immunogenetics. 1999 November; 50(3-4):213-9; Pearson et al. MHC class I-associated peptides derive from selective regions of the human genome. J Clin Invest. 2016 Dec. 1; 126(12):4690-4701; and Rock, Reits, Neefjes. Present Yourself! By MHC Class I and MHC Class II Molecules. Trends Immunol. 2016 November; 37(11):724-737).

Peptide antigens are generally known in the art. Generally, the peptide antigens in accordance with the invention are capable of binding to MHC class I proteins. It will be understood by a person skilled in the art that for each MHC class Ib molecule or polypeptide capable of presenting peptides in accordance with the invention, peptide antigens which are capable of binding to said MHC class Ib molecule or recombinant polypeptide will preferably be used. These peptide antigens can be selected based on methods known in the art.

Binding of peptide antigens to MHC class Ib molecules or to polypeptides capable of peptide antigen binding in accordance with the invention can be assessed by methods known in the art, e.g. the methods of:
Rammensee, Bachmann, Emmerich, Bachor, Stevanović. SYFPEITHI: database for MHC ligands and peptide motifs. Immunogenetics. 1999 November; 50(3-4):213-9;
Pearson et al. MHC class I-associated peptides derive from selective regions of the human genome. J Clin Invest. 2016 Dec. 1; 126(12):4690-4701; and
Rock, Reits, Neefjes. Present Yourself! By MHC Class I and MHC Class II Molecules. Trends Immunol. 2016 November; 37(11):724-737.

Such methods include experimental methods and methods for the prediction of peptide antigen binding.

Anchor residues which serve to anchor the peptide antigen on the MHC class I molecule and to ensure binding of the peptide antigen to the MHC class I molecule are known in the art.

In a preferred embodiment in accordance with all embodiments of the invention, the peptide antigen used in accordance with the invention contain any of the anchor or preferred amino acid residues in the positions as predicted for MHC class I molecules.

Such predictions can preferably be made in as described in any one of the following publications:
Rammensee et al, SYFPEITHI: database for MHC ligands and peptide motifs. Immunogenetics (1999) 50: 213-219
Nielsen et al, Protein Sci (2003) 12:1007-1017
Neefjes et al. Nat Rev Immunol. 2011 Nov. 11; 11(12):823-36
Diehl et al. Curr Biol. 1996 Mar. 1; 6(3):305-14,
Lee et al. Immunity. 1995 November; 3(5):591-600.
Desai & Kulkarni-Kale, T-cell epitope prediction methods: an overview. Methods Mol Biol. 2014; 1184:333-64.
Jumper et al. Highly accurate protein structure prediction with AlphaFold. Nature 2021; 596:583-589

In the invention, the peptide antigen is from human aquaporin 4.

It is understood that the non-anchor amino acid residues of the peptide antigen of the invention may or may not contain conservative substitutions, preferably not more than two conservative substitutions, more preferably one conservative substitution with respect to the corresponding amino acid sequence of a peptide antigen from human aquaporin 4.

Peptide antigens of the invention preferably consist of naturally occurring amino acids. However, non-naturally occurring amino acids such as modified amino acids can also be used. For instance, in one embodiment, a peptide antigen of the invention encompasses the peptidomimetic of the indicated peptide antigen amino acid sequence of human aquaporin 4.

Methods for the synthesis of peptide antigens, including peptide antigens in accordance with the invention, are well known in the art.

### Therapeutic Applications of the Invention

The recombinant polypeptides of the invention can be used for the treatment of neuromyelitis optica.

The treatment can be a treatment by inducing myelin-specific regulatory T cells. As such regulatory T cells (e.g., CD8-positive regulatory T cells) are activated in myelinated structures, they confer target cell protection against cytotoxic T cells recognizing the same or another myelin antigen. Regulatory T-cells (e.g., CD8-positive regulatory T cells) are known in the art and can be detected, for instance, by their secretion of IL-10.

While CD8-positive regulatory T cells are not as well known as CD4CD25 regulatory T cells, they have even been described to be more potent. See, for instance:
Junfeng Liu, Dacan Chen, Golay D. Nie and Zhenhua Dai CD8+CD122+ T-Cells: A Newly Emerging Regulator with Central Memory Cell Phenotypes. Front. Immunol. doi: 10.3389/fimmu.2015.00494; and
Niederlova, V., Tsyklauri, O., Chadimova, T. and Stepanek, O. (2021), CD8⁺ Tregs revisited: A heterogeneous population with different phenotypes and properties. Eur. J. Immunol., 51: 512-530. https://doi.org/10.1002/eji.202048614

While these are characterized by expression of CD122 and CD8 in mice, their human counterparts have been described to be CD8 and CXCR3 positive. See, for instance:
Shi Z, Okuno Y, Rifa'i M, Endharti A T, Akane K, Isobe K, et al. Human CD8+CXCR3+ T cells have the same function as murine CD8+CD122+ Treg. Eur J Immunol (2009) 39:2106-2119. doi:10.1002/eji.200939314).

The treatment according to the invention can be a treatment for reducing plasma or cerebrospinal fluid (CSF) levels of autoantibodies against human aquaporin 4. The human patient can be a patient who had plasma or cerebrospinal fluid (CSF) autoantibodies against human aquaporin 4 prior to the start of the treatment.

In accordance with the invention, the autoantibodies can be detected by various methods known in the art. A preferred approach are cell-based assays (CBAs) where the suspected target antigen of the autoantibodies (e.g., aquaporin 4) is overexpressed in HEK293 or CHO cells which are then incubated with serum or cerebrospinal fluid, typically for 1 h at room temperature. Mock-transfected sister cells serve as controls. Autoantibodies that bind to the cells are detected with different fluorescently labeled anti-human specific secondary antibodies that recognize total human IgG (heavy and light chain), IgG-Fc (constant chain) or IgG1. Binding is quantified by either flow cytometry (CBA-FACS) or visual scoring by microscopic evaluation of the immunofluorescence (CBA-IF), which is often titrated. Other approaches like enzyme-linked immunosorbent assays (ELISAs) or Western Blots are also possible, but often less sensitive, as conformation-sensitive antibodies may not be detected by these methods. Suitable approaches have been described in
Waters, P., Pettingill, P. & Lang, B. Detection methods for neural autoantibodies. Handb. Clin. Neurol. 133, 147-163 (2016).

Methods for the detection of Aqp4-specific autoantibodies are described in
Lennon, V. A., Kryzer, T. J., Pittock, S. J., Verkman, A. S. & Hinson, S. R. IgG marker of optic-spinal multiple sclerosis binds to the aquaporin-4 water channel. J. Exp. Med. 202, 473-477 (2005).

### Sequences

Preferred amino acid sequences referred to in the present application can be independently selected from the following sequences. The sequences are represented in an N-terminal to C-terminal order; and they are represented in the one-letter amino acid code.

Exemplary sequences which are part of the recombinant polypeptides of the invention:
Optional leader Peptide (absent from the recombinant polypeptide due to processing during cellular expression): e.g. MSRSVALAVLALLSLSGLEA (SEQ ID NO: 1)
Peptide antigen: any MHC class I peptide corresponding to MHC class I [alpha]1&2 domains, e.g. KPLPVDMVL (SEQ ID NO: 2)
First linker: For instance GGGGSGGGGSGGGGS (SEQ ID NO: 3) or GCGASGGGGSGGGGS (SEQ ID NO: 4)
beta 2 Microglobulin, for instance:

(SEQ ID NO: 5, human beta 2 Microglobulin)
IQRTPKIQVYSRHPAENGKSNFLNCYVSGFHPSDIEVDLLKNGER
IEKVEHSDLSFSKDWSFYLLYYTEFTPTEKDEYACRVNHVTLSQP
KIVKWDRDM

Second Linker, for instance:

(SEQ ID NO: 6)
GGGGSGGGGSGGGGSGGGGS

[Alpha] 1 & 2 domain derived either from human HLA-G or from any other MHC class I [alpha]1&2 domain suitable to present the selected antigenic peptide, Y84 may be C in DT variant or A
e.g. [Alpha] 1 & 2 domain derived from human HLA-G: E.g.

(SEQ ID NO: 7)
GSHSMRYFSAAVSRPGRGEPRFIAMGYVDDTQFVRFDSDSACPRM
EPRAPWVEQEGPEYWEEETRNTKAHAQTDRMNLQTLRGCYNQSEA
SSHTLQWMIGCDLGSDGRLLRGYEQYAYDGKDYLALNEDLRSWTA
ADTAAQISKRKCEAANVAEQRRAYLEGTCVEWLHRYLENGKEMLQ
RA

Or: Human HLA-A2 [alpha]1 & 2 domain: E.g.

(SEQ ID NO: 8)
GSHSMRYFFTSVSRPGRGEPRFIAVGYVDDTQFVRFDSDAASQRM
EPRAPWIEQEGPEYWDGETRKVKAHSQTHRVDLGTLRGCYNQSEA
GSHTVQRMYGCDVGSDWRFLRGYHQYAYDGKDYIALKEDLRSWTA
ADMAAQTTKHKWEAAHVAEQLRAYLEGTCVEWLRRYLENGKETLQ
RT

Human HLA-G [alpha]3 domain (or any MHC Ib [alpha]3 domain, such as HLA-F, which also interacts with ILT2 and ILT4 receptors), for instance: DPPKTHVTHHPVFDYEATLRCWALGFYPAEIILTWQRDGEDQTQDVELVETRPAGDGTFQKWAAVVVPSGE EQRYTCHVQHEGLPEPLMLRWSKEGDGGIMSVRESRSLSEDL (SEQ ID NO: 9; sequence of HLA-G [alpha]3).

Note that the following underlined amino acids of this sequence are relevant for ILT2 or ILT4 receptor interaction:

DPPKTHVTHHPVFDYEATLRCWALGFYPAEIILTWQRDGEDQTQD
VELVETRPAGDGTFQKWAAVVVPSGEEQRYTCHVQHEGLPEPLML
RWSKEGDGGIMSVRESRSLSEDL

Alternatively, a shorter form of a human HLA-G [alpha]3 domain may be used which lacks the optional C-terminal amino acid sequence from intron 4 (SKEGDGGIMSVRESRSLSEDL; SEQ ID NO: 20), i.e.:

(SEQ ID NO: 21)
DPPKTHVTHHPVFDYEATLRCWALGFYPAEIILTWQRDGEDQTQD
VELVETRPAGDGTFQKWAAVVVPSGEEQRYTCHVQHEGLPEPLML
RW,

Factor Xa restriction site: IEGRTGTKLGP (SEQ ID NO: 10)
SpotTag: PDRVRAVSHWSSC (SEQ ID NO: 11)
Myc tag: EQKLISEEDL (SEQ ID NO: 12)
His tag: HHHHHH* (SEQ ID NO: 13)
Spacer sequence: e.g. NSAVD (SEQ ID NO: 14) or GS

Further (alternative) exemplary peptide antigens which can be part of the recombinant polypeptides of the invention are as follows:
VLISLCFGL (SEQ ID NO: 22), preferably in recombinant polypeptides containing HLA-G alpha1 and 2 domains
IIGAGILYL (SEQ ID NO: 23), preferably in recombinant polypeptides containing HLA-A2 alpha1 and 2 domains
IIGAVLAGGL (SEQ ID NO: 24), preferably in recombinant polypeptides containing HLA-A2 alpha1 and 2 domains

Example for a recombinant polypeptide of the invention (with the optional leader peptide):

MSRSVALAVLALLSLSGLEAKPLPVDMVLGCGASGGGGSGGGGSI
QRTPKIQVYSRHPAENGKSNFLNCYVSGFHPSDIEVDLLKNGERI
EKVEHSDLSFSKDWSFYLLYYTEFTPTEKDEYACRVNHVTLSQPK
IVKWDRDMGGGGSGGGGSGGGGSGGGGSGSHSMRYFSAAVSRPGR
GEPRFIAMGYVDDTQFVRFDSDSACPRMEPRAPWVEQEGPEYWEE
ETRNTKAHAQTDRMNLQTLRGCYNQSEASSHTLQWMIGCDLGSDG
RLLRGYEQYAYDGKDYLALNEDLRSWTAADTAAQISKRKCEAANV
AEQRRAYLEGTCVEWLHRYLENGKEMLQRADPPKTHVTHHPVFDY
EATLRCWALGFYPAEIILTWQRDGEDQTQDVELVETRPAGDGTFQ
KWAAVVVPSGEEQRYTCHVQHEGLPEPLMLRWSKEGDGGIMSVRE
SRSLSEDLGSPDRVRAVSHWSSC*
(SEQ ID NO: 15; note that the
asterisk denotes the stop codon)

Note that the sequence of the peptide antigen (here: KPLPVDMVL) of the above full length recombinant polypeptide can be substituted by any peptide antigen sequence in accordance with the invention, i.e. by any peptide antigen presented by said recombinant polypeptide, wherein the peptide antigen is a peptide of human aquaporin 4. That is, recombinant polypeptides of the invention may consist of a sequence consisting of a peptide antigen which is a peptide of human aquaporin 4 (e.g., any one of the peptide antigens of SEQ ID NOs: 2, 22, 23, and 24), followed by the sequence of

GCGASGGGGSGGGGSIQRTPKIQVYSRHPAENGKSNFLNCYVSGF
HPSDIEVDLLKNGERIEKVEHSDLSFSKDWSFYLLYYTEFTPTEK
DEYACRVNHVTLSQPKIVKWDRDMGGGGSGGGGSGGGGSGGGGSG
SHSMRYFSAAVSRPGRGEPRFIAMGYVDDTQFVRFDSDSACPRME
PRAPWVEQEGPEYWEEETRNTKAHAQTDRMNLQTLRGCYNQSEAS
SHTLQWMIGCDLGSDGRLLRGYEQYAYDGKDYLALNEDLRSWTAA
DTAAQISKRKCEAANVAEQRRAYLEGTCVEWLHRYLENGKEMLQR
ADPPKTHVTHHPVFDYEATLRCWALGFYPAEIILTWQRDGEDQTQ
DVELVETRPAGDGTFQKWAAVVVPSGEEQRYTCHVQHEGLPEPLM
LRWSKEGDGGIMSVRESRSLSEDLGSPDRVRAVSHWSSC*
(SEQ ID NO: 16; note that the asterisk denotes
the stop codon)

These recombinant polypeptides of the invention may also contain the optional leader peptide as exemplified above.

The receptors ILT2 (also known as LILRB1) and ILT4 (also known as LILRB2) are known in the art. Preferred sequences of these receptors in accordance with the invention are as follows:

ILT2:
(SEQ ID NO: 17)
MTPILTVLICLGLSLGPRTHVQAGHLPKPTLWAEPGSVITQGSPV
TLRCQGGQETQEYRLYREKKTALWITRIPQELVKKGQFPIPSITW
EHAGRYRCYYGSDTAGRSESSDPLELVVTGAYIKPTLSAQPSPVV
NSGGNVILQCDSQVAFDGFSLCKEGEDEHPQCLNSQPHARGSSRA
IFSVGPVSPSRRWWYRCYAYDSNSPYEWSLPSDLLELLVLGVSKK
PSLSVQPGPIVAPEETLTLQCGSDAGYNRFVLYKDGERDFLQLAG
AQPQAGLSQANFTLGPVSRSYGGQYRCYGAHNLSSEWSAPSDPLD
ILIAGQFYDRVSLSVQPGPTVASGENVTLLCQSQGWMQTFLLTKE
GAADDPWRLRSTYQSQKYQAEFPMGPVTSAHAGTYRCYGSQSSKP
YLLTHPSDPLELVVSGPSGGPSSPTTGPTSTSGPEDQPLTPTGSD
PQSGLGRHLGVVIGILVAVILLLLLLLLLFLILRHRRQGKHWTST
QRKADFQHPAGAVGPEPTDRGLQWRSSPAADAQEENLYAAVKHTQ
PEDGVEMDTRSPHDEDPQAVTYAEVKHSRPRREMASPPSPLSGEF
LDTKDRQAEEDRQMDTEAAASEAPQDVTYAQLHSLTLRREATEPP
PSQEGPSPAVPSIYATLAIH
ILT4:
(SEQ ID NO: 18)
MTPIVTVLICLGLSLGPRTHVQTGTIPKPTLWAEPDSVITQGSPV
TLSCQGSLEAQEYRLYREKKSASWITRIRPELVKNGQFHIPSITW
EHTGRYGCQYYSRARWSELSDPLVLVMTGAYPKPTLSAQPSPVVT
SGGRVTLQCESQVAFGGFILCKEGEEEHPQCLNSQPHARGSSRAI
FSVGPVSPNRRWSHRCYGYDLNSPYVWSSPSDLLELLVPGVSKKP
SLSVQPGPVVAPGESLTLQCVSDVGYDRFVLYKEGERDLRQLPGR
QPQAGLSQANFTLGPVSRSYGGQYRCYGAHNLSSECSAPSDPLDI
LITGQIRGTPFISVQPGPTVASGENVTLLCQSWRQFHTFLLTKAG
AADAPLRLRSIHEYPKYQAEFPMSPVTSAHAGTYRCYGSLNSDPY
LLSHPSEPLELVVSGPSMGSSPPPTGPISTPAGPEDQPLTPTGSD
PQSGLGRHLGVVIGILVAWLLLLLLLLLFLILRHRRQGKHWTSTQ
RKADFQHPAGAVGPEPTDRGLQWRSSPAADAQEENLYAAVKDTQP
EDGVEMDTRAAASEAPQDVTYAQLHSLTLRRKATEPPPSQEREPP
AEPSIYATLAIH

The sequence of human aquaporin 4 is known in the art. Preferred amino acid sequences of human aquaporin 4 are as follows:
>sp|P55087-2|AQP4_HUMAN Isoform 1 of Aquaporin-4 OS=Homo sapiens OX=9606 GN=AQP4

(SEQ ID NO: 19)
MVAFKGVWTQAFWKAVTAEFLAMLIFVLLSLGSTINWGGTEKPLP
VDMVLISLCFGLSIATMVQCFGHISGGHINPAVTVAMVCTRKISI
AKSVFYIAAQCLGAIIGAGILYLVTPPSWVGGLGVTMVHGNLTAG
HGLLVELIITFQLVFTIFASCDSKRTDVTGSIALAIGFSVAIGHL
FAINYTGASMNPARSFGPAVIMGNWENHWIYWVGPIIGAVLAGGL
YEYVFCPDVEFKRRFKEAFSKAAQQTKGSYMEVEDNRSQVETDDL
ILKPGWHVIDVDRGEEKKGKDQSGEVLSSV

>sp|P55087|AQP4_HUMAN Aquaporin-4 Isoform 2 OS=Homo sapiens OX=9606 GN=AQP4 PE=1 SV=2

(SEQ ID NO: 29)
MSDRPTARRWGKCGPLCTRENIMVAFKGVWTQAFWKAVTAEFLAM
LIFVLLSLGSTINWGGTEKPLPVDMVLISLCFGLSIATMVQCFGH
ISGGHINPAVTVAMVCTRKISIAKSVFYIAAQCLGAIIGAGILYL
VTPPSVVGGLGVTMVHGNLTAGHGLLVELIITFQLVFTIFASCDS
KRTDVTGSIALAIGFSVAIGHLFAINYTGASMNPARSFGPAVIMG
NWENHWIYWVGPIIGAVLAGGLYEYVFCPDVEFKRRFKEAFSKAA
QQTKGSYMEVEDNRSQVETDDLILKPGVVHVIDVDRGEEKKGKDQ
SGEVLSSV

>NP_001304313.1 aquaporin-4 isoform M1x [Homo sapiens]

MSDRPTARRWGKCGPLCTRENIMVAFKGVWTQAFWKAVTAEFLAM
LIFVLLSLGSTINWGGTEKPLPVDMVLISLCFGLSIATMVQCFGH
ISGGHINPAVTVAMVCTRKISIAKSVFYIAAQCLGAIIGAGILYL
VTPPSWVGGLGVTMVHGNLTAGHGLLVELIITFQLVFTIFASCDS
KRTDVTGSIALAIGFSVAIGHLFAINYTGASMNPARSFGPAVIMG
NWENHWIYWVGPIIGAVLAGGLYEYVFCPDVEFKRRFKEAFSKAA
QQTKGSYMEVEDNRSQVETDDLILKPGWVHVIDVDRGEEKKGKDQ
SGEVLSSVXLEDRTESRQDSLELSSDFLPPIKETDLL
(SEQ ID NO: 30; where X can be tryptophan,
cysteine, arginine or serine or a stop codon)

The present invention is further illustrated by the following non-limiting examples:

### EXAMPLES

### Example 1

### Methods for Producing Recombinant Polypeptides of the Invention

Expi-293F cells (Thermo Fisher), grown in Expi-293™ expression medium (Thermo Fisher): transfection of 1 μg DNA into 2.5×10⁶ cells/ml using the Expifectamine™ 293 Transfection kit (Thermo Fisher) using Opti-MEM (Thermo Fisher) for complexation of DNA with Expifectamine, after 18-20 h, addition of enhancer according to the protocol, harvesting of the supernatant after 4-6 days (37° C., 8% CO₂, humidified incubator), 19 mm² orbital shaker 125 rpm
Spot-tag protein purification: equilibration of Spot-Cap resin: transfer of desired slurry amount into an appropriate tube, sediment beads by centrifugation (4° C., 4 min, 2500 g), remove & discard supernatant, add 10 bed volumes PBS (cold) to beads, invert to mix, sediment beads by centrifugation (4° C., 4 min, 2500 g), remove & discard supernatant, repeat 2 times
Add required volume beads to supernatant, incubate ON, 4° C. on a rotator, wash beads by repeated centrifugation (4° C., 4 min, 2500 g), and removal of supernatant
Prepare a 500 μM Spot-peptide solution in PBS, remove the supernatant, incubate with ⅓rd of the spot-peptide solution for 5-10 min

Sediment beads by centrifugation. Use Amicon Ultra-4 centrifugal filters (15 kDa cutoff) for Protein concentration and spot-peptide removal with 15 kDa Amicon cutoff columns

Rinse the Amicon Ultra-4 centrifugal filters (15 kDa cutoff) with PBS followed by 0.1 N NaOH (centrifugation at 4000 g, 4° C.) to remove trace amounts of glycerine.

### ELISPOT:

### 1) Cell Culture

### A) PBMC Isolation (Under a Laminar Flow Hood)

To isolate peripheral blood mononuclear cells (PBMC), a density centrifugation was performed with white blood cells from a leukocyte reduction chamber and density gradient medium (e.g. Ficoll, or ROTI Sep 1077). Cells were centrifuged for 20 min at 1200×g without brake followed by collection of the interphase ring that was washed with 1×PBS (5 min, 300×g). PBMC were frozen till further use.

### B) PBMC Pulsing (Under a Laminar Flow Hood)

PBMCs were thawed 1 day prior to PBMC pulsing (d-1) and kept over night in 5 ml X-VIVO 15 medium containing 5% human AB serum in a well of a 6 well plate at 37° C.

On the next day (d0) cells were counted and resuspended in X-VIVO 15 complete medium (5% hAB serum & cytokine cocktail: 20 ng/ml hIL-2, 20 ng/ml hGM-CSF, 10 ng/ml hIL-4 & 10 ng/ml hTGF-b1) at a cell density of 3×10⁶ cells/ml.

For experiments, 3×10⁶ cells were seeded in the respective wells of a 12-well plate with a final volume of 1000 μl X-VIVO complete medium with cytokine cocktail and

5 μg/ml of an AIM Bio molecule or the respective controls.

On day 3, 1 ml complete medium (with cytokines) was added, on day 6, a second pulse with 5 μg/ml of a recombinant polypeptide of the invention or a surrogate thereof (collectively referred to as “AIM Bio” molecule) was performed (after removing medium). On days 7, 10 & 12, 1 ml complete medium (with cytokines) was added.

Required:
X-VIVO 15 medium+5% human AB serum
X-VIVO 15 complete medium: X-VIVO 15 medium+2% human AB serum supplemented with cytokine cocktail: 10 ng/ml TGF-b1, 10 ng/ml IL-4, 20 ng/ml IL-2, 20 ng/ml GM-CSF

### 2) ELISPOT

### Laminar Flow Hood

On day 13, ELISPOT plates were coated using anti-hIL10 (clone 9D-7, 1:500 dilution in PBS, sterile filtered) and aIL10 (10G8-biotin) and on day 14, 200,000 cells were seeded per well on the ELISPOT plates in duplicates, including negative controls (cells plus PBS) and a positive control (e.g. LPS).

The PFDF membrane was activated with 50 μl/well EtOH (35% v/v) for 1 min followed by 5× washing with 200 μl distilled sterile water. Plate was coated with 100 μl/well antibody solution at 4° C. over night. On the next day, unbound coating antibody was removed, 5 washing steps were performed with 200 μl PBS and 200 μl blocking buffer (X-VIVO 15 5% hAB serum) was added and the plate incubated for 30 min-2 h at room temperature.

The respective antigenic peptide in DMSO or DMSO as a control were prepared, and a final amount of 5 μg peptide/ml was added to the final volume of 100 μl/well. 150,000 cells were seeded per well in X-VIVO 15 medium with 5% human AB serum. Blocking buffer (X VIVO 15 medum+5% hAB serum) was carefully removed, and medium with PBS as negative control and stimulants (5 μg/ml total volume in each well) were added to the other wells and incubated at 37° C. over night.

### Outside the Laminar Flow Hood

Secondary antibody was prepared: 1 μg/ml aIL-10-biotinylated antibody in 0.5% BSA/1×PBS (1:1000 dilution) and horseradish peroxidase-conjugated streptavidin (1:750 in 0.5% BSA/PBS), tetramethylbenzidine solution was filtered using a 0.45 μm filter and stored at 4° C. till use.

Cell supernatant was removed and 5× washed using 100 μl PBS. Last excess buffer was removed using paper towels.

25 μl diluted HRP-streptavidin (1:750) was added per well and incubated for 1 h at room temperature in the dark followed by 5 washing steps using sterile 1×PBS.

100 μl of filtered TMB substrate was added per well for 15-25 min till blue sports developed. Reaction was stopped by washing the wells thoroughly with tapped water.

Plastic underdrains of the plates was removed and the bottom and sides of the plates were washed with tap water and dried.

Plates were read out using an ImmunoSpot S6 Ultra-V Analyzer (Cellular Technology Limited), analysed in Excel and graphs/statistics were done in Graphad Prism.

Required:
Capture antibodies: anti-hIL10 (Clone: 9D-7, Mabtech #3430-3-250; 1:500 dilution), anti-hIL10-biotinylated (Mabtech, #3430-6-250)
1×PBS (sterile)
35% EtOH (v/v)
Blocking buffer: X-vivo 5% hAB serum (sterile) [blocking is done in the same medium as cell culture]
Dilution buffer: 0.5% BAS in PBS
Washing buffer: 1×PBS
Medium: for T cells, X-VIVO 15 medium (Lonza)
Filter syringe: Millex GV
ELISPOT PVDF plate (#MSIP4510, Millipore)
TMB substrate

### Example 2: Surrogates of Recombinant Polypeptides of the Invention Induce IL10 Secreting Treg in Mice

Wild type black 6 mice were injected with 100 μg recombinant polypeptides (also referred to as “AIMBio”) having the following sequences,

Ova_KbG
(SEQ ID NO: 25)
SIINFEKLGCGASGGGGSGGGGSIQRTPKIQVYSRHPAENGKSNF
LNCYVSGFHPSDIEVDLLKNGERIEKVEHSDLSFSKDWSFYLLYY
TEFTPTEKDEYACRVNHVTLSQPKIVKWDRDMGGGGSGGGGSGGG
GSGGGGSGPHSLRYFVTAVSRPGLGEPRYMEVGYVDDTEFVRFDS
DAENPRYEPRARWMEQEGPEYWERETQKAKGNEQSFRVDLRTLLG
CYNQSKGGSHTIQVISGCEVGSDGRLLRGYQQYAYDGCDYIALNE
DLKTWTAADMAALITKHKWEQAGEAERLRAYLEGTCVEWLRRYLK
NGNATLLRTDPPKTHVTHHPVFDYEATLRCWALGFYPAEIILTWQ
RDGEDQTQDVELVETRPAGDGTFQKWAAVVVPSGEEQRYTCHVQH
EGLPEPLMLRWSKEGDGGIMSVRESRSLSEDLGSPDRVRAVSHWS
SC,
and
Gp34_KbG
(SEQ ID NO: 26)
AVYNFATMGCGASGGGGGGGGSIQRTPKIQVYSRHPAENGKSNFL
NCYVSGFHPSDIEVDLLKNGERIEKVEHSDLSFSKDWSFYLLYYT
EFTPTEKDEYACRVNHVTLSQPKIVKWDRDMGGGGSGGGGSGGGG
SGGGGSGPHSLRYFVTAVSRPGLGEPRYMEVGYVDDTEFVRFDSD
AENPRYEPRARWMEQEGPEYWERETQKAKGNEQSFRVDLRTLLGC
YNQSKGGSHTIQVISGCEVGSDGRLLRGYQQYAYDGCDYIALNED
LKTWTAADMAALITKHKWEQAGEAERLRAYLEGTCVEWLRRYLKN
GNATLLRTDPPKTHVTHHPVFDYEATLRCWALGFYPAEIILTWQR
DGEDQTQDVELVETRPAGDGTFQKWAAVVVPSGEEQRYTCHVQHE
GLPEPLMLRWSKEGDGGIMSVRESRSLSEDLGSPDRVRAVSHWSS
C,

for inducing tolerance towards an OVA peptide or an viral Gp34 peptide, respectively. The Gp34 peptide is a well-characterized T cell epitope derived from Lymphocytic Choriomeningitis virus (LCMV) Glycoprotein. While this antigen was traditionally named Gp33, the epitope presented on H2-K^{b} was later found to comprise just amino acids 34-41. (An epitope beginning at amino acid 33 is, in contrast, presented on H2-K^{d}.) Therefore, we call the H2-K^{b} epitope Gp34, which is in line with the most recent recommendations. Still, there is an ambiguous use of the Gp33 and Gp34 nomenclature in the literature. The first 8 amino acids of SEQ ID NO: 26 show the correct sequence. After 2 weeks, mice were sacrificed, and splenocytes re-challenged either with the matching or a mismatching peptide. IL-10 secreting cells were quantified by ELIspot. The results are shown in FIG. 3.

### Example 3: Surrogates of Recombinant Polypeptides of the Invention Selectively Prevent CD8+ T-Cell Driven EAE in Mice

As described in (Na et al, Brain. 2008 September; 131(Pt 9):2353-65.), the adoptive transfer of CD8⁺ OT-I T cells that recognize an ovalbumin epitope in the context of H2-K^{b} into mice which express ovalbumin in oligodendrocytes leads to experimental autoimmune encephalomyelitis which recapitulates many MS and late stage NMO symptoms. In this animal model, a single injection of 500 μg of recombinant polypeptides surrogate molecules (also referred to as “AIMBio”) that induce tolerance towards the targeted ovalbumin epitope almost completely prevented EAE symptoms, while a surrogate molecule presenting a control peptide hat no significant protective effects (FIG. 4). The sequences of the recombinant polypeptide surrogate molecules are listed in Example 2.

### Example 4: Some Surrogates of Recombinant Polypeptides of the Invention Selectively Prevent CD4⁺ T Cell Driven EAE in Mice

At the day of the 33 μg or 100 μg recombinant polypeptide of the invention surrogate molecule (“AIM Bio”) i.p. injection, 100 μl MOG35-55 peptide/CFA (Complete Freund's Adjuvance; final concentration Mycobacterium tuberculosis H37RA and peptide each 1 mg/ml) emulsion were injected each left and right s.c. into the flank and 250 ng pertussis toxin (in 200 μl PBS) intraperitoneally. A second pertussis toxin injection was given 3 days later. In this animal model, a single injection AIM Bio surrogate molecules that induce tolerance towards the a Mog epitope (Mog44_Kb_G) significantly reduced EAE symptoms, while a surrogate molecule presenting a control peptide (Gp34) or a non-functional Mog peptide (Mog37) hat no significant protective effects (FIG. 5). In this model Mog44 AIM Bio also prevented inflammation and CD8 T cell infiltration in the spinal cord (FIG. 6). The sequences of the recombinant polypeptide surrogate molecules were listed in example 2 or as follows:

Mog44_DbG
(SEQ ID NO: 27)
FSRVVHLYRNGGCGASGGGGSGGGGSIQRTPKIQVYSRHPAENGK
SNFLNCYVSGFHPSDIEVDLLKNGERIEKVEHSDLSFSKDWSFYL
LYYTEFTPTEKDEYACRVNHVTLSQPKIVKWDRDMGGGGSGGGGS
GGGGSGGGGSGPHSMRYFETAVSRPGLEEPRYISVGYVDNKEFVR
FDSDAENPRYEPRAPWMEQEGPEYWERETQKAKGQEQWFRVSLRN
LLGCYNQSAGGSHTLQQMSGCDLGSDWRLLRGYLQFAYEGRDYIA
LNEDLKTWTAADMAAQITRRKWEQSGAAEHYKAYLEGECVEWLHR
YLKNGNATLLRTDPPKTHVTHHPVFDYEATLRCWALGFYPAEIIL
TWQRDGEDQTQDVELVETRPAGDGTFQKWAAVVVPSGEEQRYTCH
VQHEGLPEPLMLRWSKEGDGGIMSVRESRSLSEDLGSPDRVRAVS
HWSSC
Mog37_DbG
(SEQ ID NO: 28)
VGWYRSPFSRGCGASGGGGSGGGGSIQRTPKIQVYSRHPAENGKS
NFLNCYVSGFHPSDIEVDLLKNGERIEKVEHSDLSFSKDWSFYLL
YYTEFTPTEKDEYACRVNHVTLSQPKIVKWDRDMGGGGSGGGGGG
GGSGGGGSGPHSMRYFETAVSRPGLEEPRYISVGYVDNKEFVRFD
SDAENPRYEPRAPWMEQEGPEYWERETQKAKGQEQWFRVSLRNLL
GCYNQSAGGSHTLQQMSGCDLGSDWRLLRGYLQFAYEGRDYIALN
EDLKTWTAADMAAQITRRKWEQSGAAEHYKAYLEGECVEWLHRYL
KNGNATLLRTDPPKTHVTHHPVFDYEATLRCWALGFYPAEIILTW
QRDGEDQTQDVELVETRPAGDGTFQKWAAVVVPSGEEQRYTCHVQ
HEGLPEPLMLRWSKEGDGGIMSVRESRSLSEDLGSPDRVRAVSHW
SSC

In this model Mog44 AIM Bio also completely prevented the formation of MOG-specific autoantibodies in the serum as tested by ELISA (FIG. 7; see also the confirmation by FIG. 10). This is a strong indicator that the recombinant polypeptides of the invention are effective therapeutics in NMO, which are often characterized by antibody responses against human aquaporin 4. Thus, the patient population is defined by a common autoimmune-related antigen. Certain MHC molecules are also associated with NMO.

Mog-reactive antibodies in sera of AIM Bio (33 or 100 μg) treated mice were detected via standard ELISA protocol, with 3 washes in between each step. Briefly, ELISA plates were coated with 10 μg/ml Mog35-55 peptide, blocked with PBS 1% BSA, before mouse sera diluted 1:25 in PBS 1% BSA were added for 1 h. Anti-mouse IgG-HRP or anti-mouse heavy and light chain—HRP antibodies diluted 1:5000 were used for detection.

### Example 5: Human Recombinant Polypeptide Candidates of the Invention for NMO

The recombinant polypeptides of the invention are newly developed protein complexes derived from the pregnancy-associated immunosuppressive MHC molecule HLA-G. It is likely that HLA-G enables an embryo to influence the maternal immune system to tolerate embryonic antigens but further antagonize antigens from pathogens. The recombinant polypeptides of the invention containing variable peptides were able to selectively eliminate peptide-specific cytotoxic effector T cells as well as induce peptide-specific regulatory T cells in the test tube.

FIG. 8 shows a list of the human MS & MOGAD recombinant polypeptide candidates.

The inventors' findings show that single-chain proteins containing AQP4 peptide antigens and a HLA-G alpha 3 domain can induce tolerogenic T cells in healthy donors. Thus, CD8 Treg were upregulated by at least 30% in 65% of all healthy blood donors (FIG. 9).

### Example 6: Further Proof-of-Principle of Stability and Effects of the Recombinant Polypeptides of the Invention

Additionally, the inventors set out to obtain and test recombinant polypeptides having the general structure of the recombinant polypeptides of the invention but containing various different peptide antigens, in order to obtain further proof-of-principle that recombinant polypeptides of the invention and surrogates thereof are stable and efficacious. As shown in FIGS. 11 and 12, respectively, the tested recombinant polypeptides are stable during freeze-thawing and storage and are thermally stable. Further, they induce Treg in a dose-dependent manner (FIG. 13) and inhibit T cell lysis in a dose-dependent manner (FIG. 14). Effects of the recombinant polypeptides on the serum cytokine profile in EAE-ODC Ova mice are shown in FIG. 15. There is an induction of IL-10 and possibly IL-4, both known to be immunosuppressive cytokines downregulating immune responses in inflammatory settings. This requires an HLA-G alpha3 domain plus a cognate peptide. IL-2 seems to be induced in response to presenting the cells with a cognate peptide that is irrespective of the alpha3 domain. IL-2 is needed for T cell activation and survival.

### Example 7: Therapeutic Effect of Recombinant Peptides, which Comprise a Murine Aquaporin Antigen Peptide, in 2D2 TCR Transgenic Mice

### Materials and Methods

### Experimental Models

2D2 TCR transgenic mice were divided into 4 cohorts. Each cohort consisted of at least 5 animals. WT (C57BL/6) littermate-matched mice were in the 5^{th} cohort. Experiment period started when the 2D2 mice spontaneously developed optic neuritis. Disease onset was determined either by optical abnormality or by elevated levels of anti-MOG IgG in serum. On the day following disease onset, mice were injected (i.v) with either one of treatment mouse-adapted single-chain MHC Ib molecules AQP147_KbG (antigen peptide: VTTVHGNL; SEQ ID NO: 39)/AQP203_KbG (antigen peptide: FAINYTGASM; SEQ ID NO: 40) or with control AIM Bio Gp34_KbG. Control mice received PBS only. Dose was set at 5 mg/kg of body weight in 250 μl. control or treatment injections were administered intravenously (DO). Untreated mice received PBS. The injections were repeated on D15 and D30. Mice were weighed daily and EAE symptoms were monitored (Table 1). Mice reaching an EAE score of 6 were sacrificed prior to completion of study period. All animals were sacrificed on D42 with CO₂. Optic nerve, eyes, spinal cord, brain, serum, and lymph nodes were harvested and cryo-preserved in −20° C. Heart blood was collected and processed for serum analysis.
EAE was scored according to a 10 point scoring system (Bittner et al., Journal of visualized experiments: JoVE, (86), 51275)

### Immunohistochemistry

20 μm thick serial sections of eyes were cut and stained with hematoxylin and eosin. Briefly, cryosections were dried for 10 min at room temperature and stained for 10 min in hematoxylin staining solution. Tissue sections were washed for 10 min under running water and stained for 30 sec in Eosin-staining solution. Sections were then sequentially dehydrated for 30 sec each in 70%, 96% and 100% ethanol. Following this, sections were incubated with xylol for 10 min and mounted with vitro cloud.

### Immunofluorescence

Serial, 10 μm thick longitudinal sections of optic nerve were cut for immunohistochemistry. Tissue sections were fixed with 4% PFA in PBS for 10 minutes; followed by blocking with buffer containing 5% BSA, 0.2% Triton-X100 and 5% NGS in 1×PBS for 2 hours at RT. Tissues were stained overnight at 4° C. with either single or a suitable combination of primary antibodies in staining buffer containing 1% BSA, 1% NGS and 0.2% Triton-X100. The primary antibodies are: i) rat anti-mouse CD3 (1:200, Invitrogen); ii) rat anti-mouse CD8 (1:200, Biorad); iii) rabbit anti-cleaved-caspase3 (1:400, Cell signaling). Sections were washed thrice with PBS and stained for 1 h in dark at RT with corresponding fluorescently-labelled secondary antibodies: i) anti-rabbit-Cy3 (1:300, Dianova); ii) anti-rat-AF488 (1:300, Invitrogen). Sections were washed with PBS and DNA was stained with DAPI (1:500, Sigma-Aldrich) for 10 min at RT in dark. Sections were finally washed and mounted with aquapolymount (Polysciences). Optical sections were obtained using Zeiss Axiocam at 20× or 40× magnification.

### Quantification of Immunostaining

Immunofluorescent images were quantified using ImageJ-Fiji Version 1.53t. 3-4 sections were analyzed for each animal. Images were split into individual channels and converted to 8-bit images. Threshold for 8-bit images of cleaved caspase-3 staining was set at 5 MFI and from DAPI staining was set at 100. Area covered by cleaved-caspase 3/Area covered by DAPI gives the percentage of cleaved caspase3 in a section.

### Quality Control and Stability

After purification of the single chain MHC Ib molecules, their stability was analysed after 1 and 3 freeze-thawing cycles, storage for 5 days at room temperature and heating up to a temperature of 50° C. for 30 min. For this, A) a Coomassie gel staining of a 12% polyacrylamide gel using 2 μg single chain MHC-Ib molecule and B) an [symbol] HLA-G Western blot using the 2A12aHLA-G antibody (1:1000) blot using 1 μg protein was performed under non-reducing conditions. Both monomers and dimers are detectable.

### Results

### Quality Control and Stability

Quality control and stability tests showed that both NMO surrogate single-chain MHC Ib molecules are very stable (FIGS. 17-19). However, AQP203_H2KbG_spt seems to multimerize more, which could explain why it is less effective in some assays.

### Therapeutic Effects

Remarkably, treatment with AQP147_KbG conferred complete protection from EAE in treated mice (FIG. 20), inhibited immune cell infiltration in optic nerve (FIG. 21) and completely inhibited apoptosis in optic nerve, spinal cord and retina (FIG. 22). Further, while there were less CD8 T cells in AIM treated mice, there were few, but clearly more regulatory T cells in the optic nerve (FIG. 23). Additionally, AQP147_KbG completely rescued IRL (inner retinal layer) cells from degradation (FIG. 24).

These data confirm that the recombinant polypeptides of the invention and surrogates thereof have therapeutic efficacy. Thus, according to the invention, the recombinant polypeptides of the invention can be used in the treatment of neuromyelitis optica in a human patient.

### INDUSTRIAL APPLICABILITY

The pharmaceutical compositions, polypeptides, nucleic acids, cells, and products for use in the invention are industrially applicable. For example, they can be used in the manufacture of, or as, pharmaceutical products.

1. A recombinant polypeptide capable of presenting a peptide antigen, the recombinant polypeptide comprising, in an N- to C-terminal order,
i) a peptide antigen presented by said recombinant polypeptide, wherein the peptide antigen is a peptide of human aquaporin 4;
ii) optionally a linker sequence;
iii) optionally a sequence of a human polypeptide domain comprising a sequence of a human β2 microglobulin, or an amino acid sequence at least 90% identical to the amino acid sequence of human β2 microglobulin represented by SEQ ID NO: 5;
iv) optionally a linker sequence;
v) optionally an [alpha] 1 domain of an MHO molecule;
vi) optionally an [alpha] 2 domain of an MHO molecule;
vii) an [alpha] 3 domain of an MHO class Ib molecule or a derivative of an [alpha] 3 domain of an MHO class Ib molecule, said derivative being capable of binding to ILT2 or ILT4;
viii) optionally a protease cleavage site;
ix) optionally a spacer sequence; and
x) optionally an affinity tag.

2. The recombinant polypeptide according to claim 1, wherein said peptide antigen according to i) is 7 to 11 amino acids in length, preferably 8-10 amino acids in length.

3. The recombinant polypeptide according to claim 1 or 2, wherein said peptide antigen according to i) consists of an amino acid sequence selected from the group consisting of the amino acid sequences of SEQ ID NOs: 2, 22, 23 and 24.

4. The recombinant polypeptide according to any one of the preceding claims, wherein said [alpha]1 domain according to (v) and said [alpha]2 domain according to (vi) are from a human MHC class Ia molecule, preferably a human HLA-A2 molecule, or from a human MHC class Ib molecule, preferably a human HLA-G molecule.

5. The recombinant polypeptide according to any one of the preceding claims, wherein the [alpha] 3 domain of the MHC class Ib molecule according to (vii) is an [alpha] 3 domain of human HLA-E, human HLA-F or human HLA-G, preferably human HLA-G.

6. The recombinant polypeptide according to any one of the preceding claims, wherein the [alpha]3 domain or derivative according to (vii) is identical to or has at least 80% amino acid sequence identity, at least 90% amino acid sequence identity, at least 92% amino acid sequence identity, at least 94% amino acid sequence identity, at least 96% amino acid sequence identity, at least 98% amino acid sequence identity, or at least 99% amino acid sequence identity with the [alpha]3 domain having the amino acid sequence of SEQ ID NO: 9 or SEQ ID NO: 21 or is identical to the [alpha]3 domain having the amino acid sequence of SEQ ID NO: 9 or SEQ ID NO: 21.

7. The recombinant polypeptide according to any one of the preceding claims, wherein the linker sequence according to (ii) and/or the linker sequence according to (iv) comprises the amino acid sequence (GGGGS)n, wherein n is an integer equal to or higher than 1, and wherein n is an integer selected from the group consisting of 1, 2, 3, 4, 5, 6, 7, 8, 9 and 10 and is preferably selected from the group consisting of 2, 3, 4 and 5.

8. The recombinant polypeptide according to any one of the preceding claims, wherein said sequence of a human polypeptide domain according to (III) is at least 95% identical to the amino acid sequence of SEQ ID NO: 5, preferably at least 98% identical to the amino acid sequence of SEQ ID NO: 5 and more preferably identical to the amino acid sequence of SEQ ID NO: 5.

9. The recombinant polypeptide according to any one of the preceding claims, wherein said polypeptide is dimeric or multimeric.

10. The recombinant polypeptide according to any one of the preceding claims, wherein the polypeptide comprises or consists of all of the components i) to vii), wherein the polypeptide does not comprise components viii) to x), or wherein the polypeptide comprises or consists of all of the components i) to x).

11. The recombinant polypeptide according to any one of the preceding claims, further comprising an N-terminal secretion signal peptide sequence.

12. The recombinant polypeptide according to any one of claims 1-10, wherein the recombinant polypeptide consists of an amino acid sequence consisting of the following ((a) and (b)) in an N- to C-terminal order:
(a) a peptide antigen selected from the group consisting of the amino acid sequences of SEQ ID NOs: 2, 22, 23 and 24, and
(b) the amino acid sequence of SEQ ID NO: 16.

13. The recombinant polypeptide according to any one of the preceding claims, wherein the recombinant polypeptide is soluble.

14. A nucleic acid encoding one or more polypeptides according to any one of the preceding claims, wherein the nucleic acid is preferably a vector.

15. A pharmaceutical composition or kit comprising at least one nucleic acid according to claim 14.

16. A pharmaceutical composition or kit comprising at least one recombinant polypeptide according to any one of claims 1-13.

17. The pharmaceutical composition or kit according to claim 16, wherein the pharmaceutical composition or kit comprises at least two different recombinant polypeptides according to any one of claims 1-13, and wherein each of the different polypeptides comprises a different peptide antigen as defined in claim 3.

18. A pharmaceutical composition or kit according to any one of claims 15-17, for use in the treatment of neuromyelitis optica in a human patient.

19. The pharmaceutical composition or kit for use according to claim 18, wherein the treatment is treatment by immunotherapy, and wherein the treatment is preferably by inducing immunological tolerance against human aquaporin 4.

20. The pharmaceutical composition or kit for use according to any one of claims 18-19, wherein the treatment is for reducing plasma or cerebrospinal fluid levels of autoantibodies against human aquaporin 4, and wherein the human patient is a patient who had plasma or cerebrospinal fluid autoantibodies against human aquaporin 4 prior to the start of the treatment.

21. The pharmaceutical composition or kit for use according to any one of claims 18-20, wherein the treatment is by inducing myelin-specific regulatory T cells.

22. A recombinant host cell comprising a nucleic acid or a vector according to claim 14 and expressing the recombinant polypeptide according to any one of claims 1-13.

23. A method for obtaining pharmaceutical composition comprising a polypeptide according to any one of claims 1-13, the method comprising the steps of (a) culturing the recombinant host cell of claim 22 under conditions allowing expression of the recombinant polypeptide from the nucleic acid molecule, (b) recovering the recombinant polypeptide, (c) purifying the recombinant polypeptide, and (d) formulating the recombinant polypeptide into a pharmaceutical composition.


SEQUENCE LISTING (40 sequences)

SEQ ID NO: 1 - protein, 20 aa, synthetic construct
  REGION 1..20: note = Leader peptide
MSRSVALAVLALLSLSGLEA

SEQ ID NO: 2 - protein, 9 aa, synthetic construct
  REGION 1..9: note = Peptide antigen
KPLPVDMVL

SEQ ID NO: 3 - protein, 15 aa, synthetic construct
  REGION 1..15: note = First Linker
GGGGSGGGGSGGGGS

SEQ ID NO: 4 - protein, 15 aa, synthetic construct
  REGION 1..15: note = First Linker
GCGASGGGGSGGGGS

SEQ ID NO: 5 - protein, 99 aa, synthetic construct [sequence omitted]

SEQ ID NO: 6 - protein, 20 aa, synthetic construct
  REGION 1..20: note = Second Linker
GGGGSGGGGSGGGGSGGGGS

SEQ ID NO: 7 - protein, 182 aa, synthetic construct
  REGION 1..182: note = [Alpha] 1 & 2 domain derived from human HLA-G
GSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRA

SEQ ID NO: 8 - protein, 182 aa, synthetic construct
  REGION 1..182: note = Human HLA-A2 [alpha]1 & 2 domain
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRT

SEQ ID NO: 9 - protein, 113 aa, synthetic construct
  REGION 1..113: note = HLA-G [alpha]3
DPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 10 - protein, 11 aa, synthetic construct
  REGION 1..11: note = Factor Xa restriction site
IEGRTGTKLGP

SEQ ID NO: 11 - protein, 13 aa, synthetic construct
  REGION 1..13: note = SpotTag
PDRVRAVSHWSSC

SEQ ID NO: 12 - protein, 10 aa, synthetic construct
  REGION 1..10: note = Myc tag
EQKLISEEDL

SEQ ID NO: 13 - protein, 6 aa, synthetic construct
  REGION 1..6: note = His tag
HHHHHH

SEQ ID NO: 14 - protein, 5 aa, synthetic construct
  REGION 1..5: note = Spacer sequence
NSAVD

SEQ ID NO: 15 - protein, 473 aa, synthetic construct
  REGION 1..473: note = Recombinant polypeptide
MSRSVALAVLALLSLSGLEAKPLPVDMVLGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 16 - protein, 444 aa, synthetic construct
  REGION 1..444: note = Recombinant polypeptide
GCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 17 - protein, 650 aa, synthetic construct
  REGION 1..650: note = Receptor ILT2 (also known as LILRB1)
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 18 - protein, 598 aa, synthetic construct
  REGION 1..598: note = Receptor ILT4
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 19 - protein, 301 aa, synthetic construct
  REGION 1..301: note = Human aquaporin 4
MVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 20 - protein, 21 aa, synthetic construct
  REGION 1..21: note = Human HLA-G [alpha]3 C-terminal amino acid sequence from intron 4
SKEGDGGIMSVRESRSLSEDL

SEQ ID NO: 21 - protein, 92 aa, synthetic construct
  REGION 1..92: note = Human HLA-G [alpha]3 without C-terminal amino acid sequence from intron 4
DPPKTHVTHHPVFDYEATLRCWALGFYPAEIILTWQRDGEDQTQDVELVETRPAGDGTFQKWAAVVVPSGEEQRYTCHVQHEGLPEPLMLRW

SEQ ID NO: 22 - protein, 9 aa, synthetic construct
  REGION 1..9: note = Peptide antigen
VLISLCFGL

SEQ ID NO: 23 - protein, 9 aa, synthetic construct
  REGION 1..9: note = Peptide antigen
IIGAGILYL

SEQ ID NO: 24 - protein, 10 aa, synthetic construct
  REGION 1..10: note = Peptide antigen
IIGAVLAGGL

SEQ ID NO: 25 - protein, 452 aa, synthetic construct
  REGION 1..452: note = Recombinant polypeptide
SIINFEKLGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 26 - protein, 452 aa, synthetic construct
  REGION 1..452: note = Recombinant polypeptide
AVYNFATMGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 27 - protein, 455 aa, synthetic construct
  REGION 1..455: note = Recombinant polypeptide
FSRVVHLYRNGGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGPHSMRYFETAVSRPGLEEPRYISVGYVDNKEFVRFDSDAENPRYEPRAPWMEQEGPEYWERETQKAKGQEQWFRVSLRNLLGCYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 28 - protein, 454 aa, synthetic construct
  REGION 1..454: note = Recombinant polypeptide
VGWYRSPFSRGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGPHSMRYFETAVSRPGLEEPRYISVGYVDNKEFVRFDSDAENPRYEPRAPWMEQEGPEYWERETQKAKGQEQWFRVSLRNLLGCYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 29 - protein, 323 aa, synthetic construct
  REGION 1..323: note = Human aquaporin 4
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 30 - protein, 352 aa, synthetic construct
  REGION 1..352: note = Human aquaporin 4
  SITE 324: note = Xaa can be tryptophan, cysteine, arginine or serine or a stop codon
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSVXLEDRTESRQDSLELSSDFLPPIKETDLL

SEQ ID NO: 31 - protein, 9 aa, synthetic construct
  REGION 1..9: note = Peptide antigen
FLAMLIFVL

SEQ ID NO: 32 - protein, 9 aa, synthetic construct
  REGION 1..9: note = Peptide antigen
MLIFVLLSL

SEQ ID NO: 33 - protein, 8 aa, synthetic construct
  REGION 1..8: note = Peptide antigen
PLPVDMVL

SEQ ID NO: 34 - protein, 10 aa, synthetic construct
  REGION 1..10: note = Peptide antigen
AIIGAGILYL

SEQ ID NO: 35 - protein, 9 aa, synthetic construct
  REGION 1..9: note = Peptide antigen
AGHGLLVEL

SEQ ID NO: 36 - protein, 8 aa, synthetic construct
  REGION 1..8: note = Peptide antigen
KAVTAEFL

SEQ ID NO: 37 - protein, 10 aa, synthetic construct
  REGION 1..10: note = Peptide antigen
VTPPSVVGGL

SEQ ID NO: 38 - protein, 472 aa, synthetic construct
  REGION 1..472: note = Recombinant polypeptide
MSRSVALAVLALLSLSGLEAVTTVHGNLGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSPDRVRAVSHWSSC

SEQ ID NO: 39 - protein, 8 aa, synthetic construct
  REGION 1..8: note = Peptide antigen
VTTVHGNL

SEQ ID NO: 40 - protein, 10 aa, synthetic construct
  REGION 1..10: note = Peptide antigen
FAINYTGASM